(19) United States
(12) Patent Application Publication
(10) Pub. No.: US 2026/0294810 A1
(43) Pub. Date: Oct. 1, 2026

(54) IONIZABLE LIPID COMPOUND, LIPID CARRIER COMPRISING SAME, AND APPLICATION

(71) Applicant: INNOVEC BIOTHERAPEUTICS, BEIJING (CN)

(72) Inventors: Xinghua PAN, Beijing (CN); Cheng WANG, Beijing (CN)

(21) Appl. No.: 19/474,280

(22) PCT Filed: Apr. 9, 2024
(86) PCT No.: PCT/CN2024/086792

§ 371 Date: Oct. 10, 2025

(30) Foreign Application Priority Data
Apr. 12, 2023 (WO) PCT/CN2023/087734

(51) Int. Cl. A61K 9/1272; A61K 9/51; A61K 31/7105; A61K 31/711; C07C 271/20; C12N 15/88

(52) U.S. Cl. CPC A61K 9/1272; A61K 9/5123; A61K 31/7105; A61K 31/711; C07C 271/20; C12N 15/88

(57) ABSTRACT

The present invention relates to an ionizable lipid compound, a lipid carrier comprising same, and an application of the ionizable lipid compound. The present invention provides a series of compounds as shown in formula (1), a lipid carrier comprising same as an ionizable lipid, a nucleic acid lipid nanoparticle composition, and a preparation thereof. Lipid nanoparticles formed by the ionizable lipid can deliver nucleic acid molecules into cells, thus increasing the transfer rate of the nucleic acid molecules, and thereby improving the treatment effect of a nucleic acid drug.

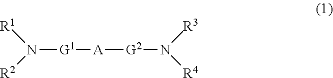

### TECHNICAL FIELD

The present disclosure belongs to the field of biological medicine, and specifically relates to an ionizable lipid compound and a lipid carrier comprising the same, a nucleic acid lipid nanoparticle composition, and a pharmaceutical preparation.

### BACKGROUND

Lipid nanoparticles are widely used in the field of drug delivery. Of these, ionizable lipids are not only excellent carriers for protein/polypeptide antigens, but also a novel type of immune adjuvant, which can directly activate antigen-presenting cells and enhance vaccine-induced immune responses. Therefore, ionizable lipids are widely used in the field of vaccines to encapsulate and transport nucleic acid molecules. Ionizable lipid is the most crucial component for enabling the targeting capability and delivery performance of lipid nanoparticles. It binds to negatively charged nucleic acids, facilitates endosomal escape and in vivo transfection of nucleic acid molecules, and exhibits many characteristics such as pH sensitivity. Ionizable lipids determine the delivery efficiency and transfection efficiency of the lipid nanoparticle delivery system. Therefore, an ionizable lipid designed with good targeting capability and delivery performance is a key element indispensable for lipid nanoparticles.

DLin-MC3-DMA (hereinafter referred to as MC3), as the most commonly-used novel ionizable lipid on the market at present, boasts the advantage of “low toxicity and high efficiency” and is the first ionizable lipid applied globally to siRNA liposome products (Onpattro). With the launch of Onpattro in the United States, MC3 and novel ionizable lipids have attracted more attention from scientists. Compared with common ionizable lipids such as DOTAP, DOTMA and DC-CHOL, such novel ionizable lipids as MC3 have lower toxicity and significantly improved drug loading capacity and safety.

There are currently four siRNA and mRNA drugs worldwide (including those under Emergency Use Authorization) that have been authorized by FDA, in all of which novel ionizable lipids are employed. Therefore, novel ionizable lipids holds broad application prospects in nucleic acid drug delivery.

### SUMMARY

The present disclosure provides a series of compounds represented by formula (1), a lipid carrier comprising the same as an ionizable lipid, a nucleic acid lipid nanoparticle composition, and a preparation thereof. The lipid nanoparticle formed of this ionizable lipid is capable of delivering nucleic acid molecules into cells, improving the transport efficiency of the nucleic acid molecules, and thus improving the therapeutic effect of the nucleic acid molecules.

In the first aspect, the present disclosure provides a compound represented by formula (1) or a pharmaceutically acceptable salt thereof:

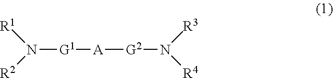

wherein
A is —O—CO—NH— or —NH—CO—O—;
G¹ and G² are independently C₁₋₄ alkylene;
R¹ and R² are independently -G³-O—C(═O)—R⁵, -G³-C(═O)—O—R⁵, -G³-NH—C(═O)—O—R⁵, -G³-NH—O—C(═O)—R⁵, -G³-NH—C(═O)—R⁵ or -G³-O—C(═O)—NH—R⁵; G³ is independently C₂₋₁₀ alkylene;
R⁵ is C₂₋₃₀ alkyl or C₂₋₃₀ alkenyl, and said alkyl or alkenyl is optionally substituted with one or more of —O—C₁₋₃₀ alkyl, —C(═O)—O—C₁₋₃₀ alkyl, —O—C(═O)—C₁₋₃₀ alkyl, —O—C₂₋₃₀ alkenyl, —C(═O)—O—C₂₋₃₀ alkenyl or —O—C(═O)—C₂₋₃₀ alkenyl; and
R³ and R⁴ are independently C₁₋₈ alkyl.

In some embodiments, G¹ and G² are independently —CH₂—, —CH₂CH₂—, —CH₂CH₂CH₂— or —CH₂CH₂CH₂CH₂—; preferably, G¹ and G² are independently —CH₂CH₂— or —CH₂CH₂CH₂—.

In some embodiments, R¹ and R² are independently -G³-O—C(═O)—R⁵, -G³-C(═O)—O—R⁵, -G³-NH—C(═O)—O—R⁵ or -G³-O—C(═O)—NH—R⁵.

In some embodiments, G³ is independently C₂₋₈ alkylene; preferably, G³ is independently

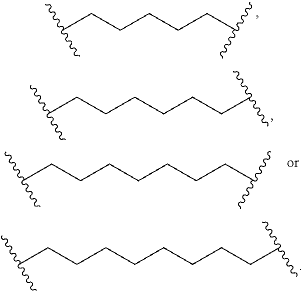 [SMILES: *CCCCC* ; *CCCCCC* ; *CCCCCCC* ; *CCCCCCCC*]

In some embodiments, R⁵ is C₂₋₂₀ alkyl or C₂₋₂₀ alkenyl, and said alkyl or alkenyl is optionally substituted with one or more of —O—C₁₋₂₀ alkyl, —C(═O)—O—C₁₋₂₀ alkyl, —O—C(═O)—C₁₋₂₀ alkyl, —O—C₂₋₂₀ alkenyl, —C(═O)—O—C₂₋₂₀ alkenyl or —O—C(═O)—C₂₋₂₀ alkenyl.

In some embodiments, R⁵ is C₂₋₂₀ alkyl or C₂₋₂₀ alkenyl, and said alkyl or alkenyl is optionally substituted with one or more of —O—C₁₋₂₀ alkyl, —C(═O)—O—C₁₋₂₀ alkyl or —C(═O)—O—C₂₋₂₀ alkenyl.

In some embodiments, R⁵ is

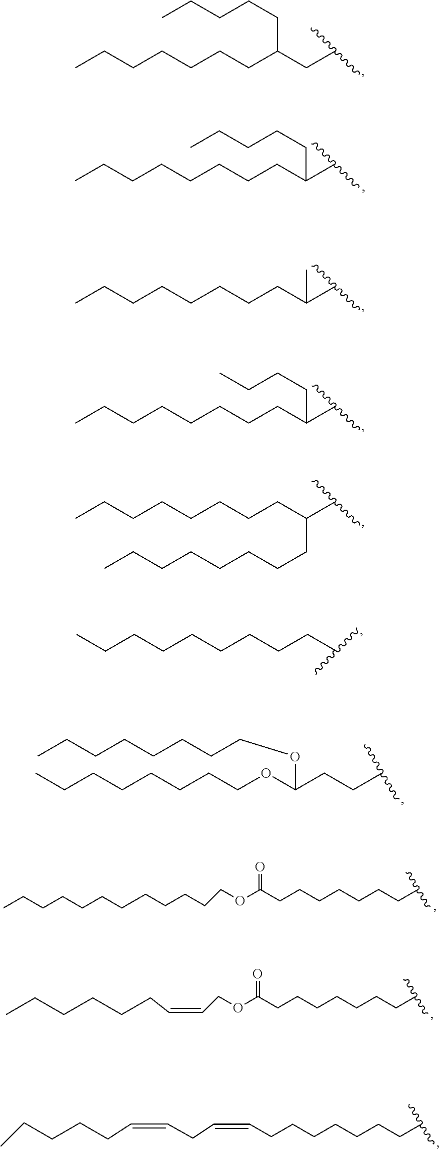

In some embodiments R¹ and R² are independently

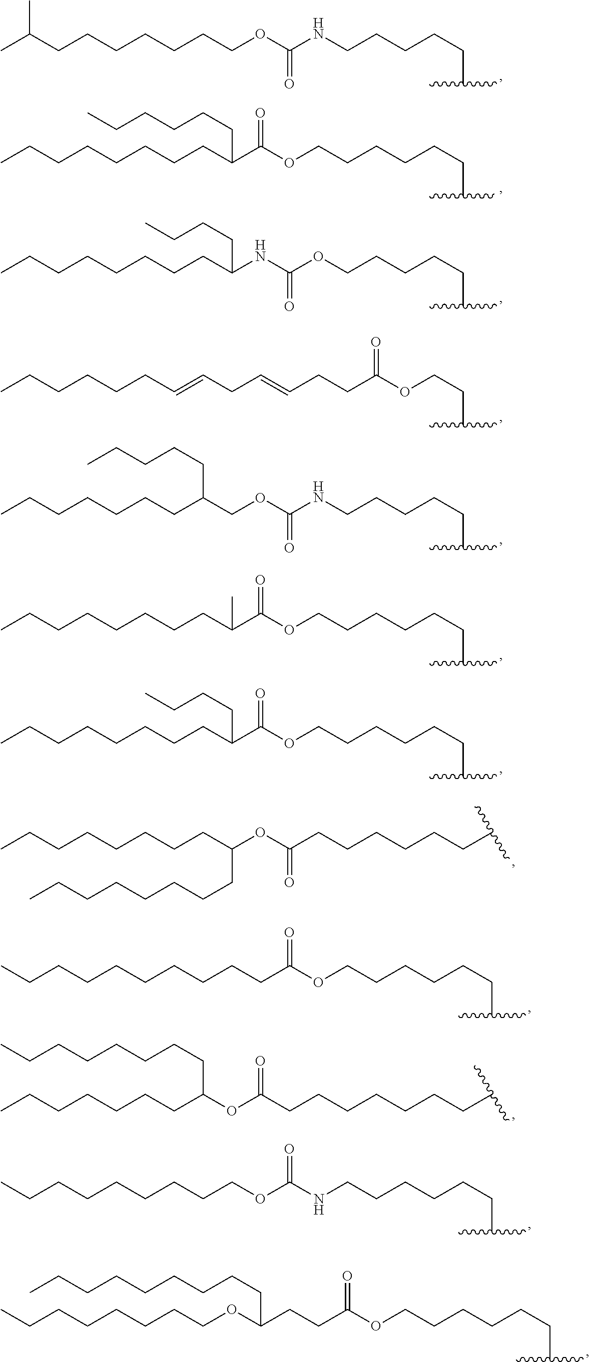

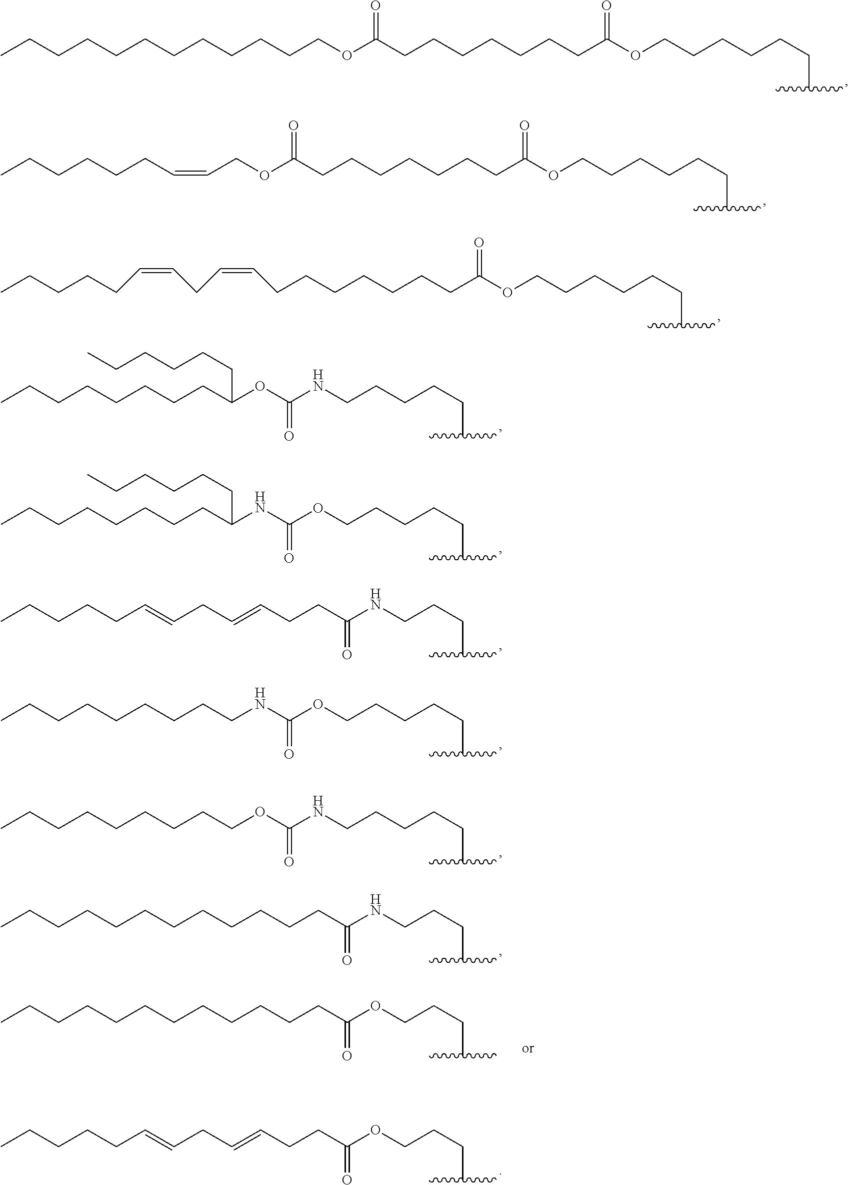

In some embodiments, R³ and R⁴ are independently C₁₋₆ alkyl; preferably, R³ and R⁴ are independently C₁₋₄ alkyl; preferably, R³ and R⁴ are independently methyl or ethyl.

The present disclosure further provides a compound shown below or a pharmaceutically acceptable salt thereof:

| | Structure of Compound |
|---|---|
| 1 | 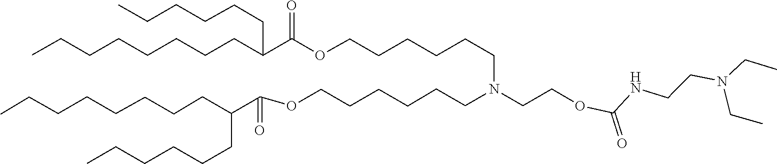 [SMILES: CCCCCCCCC(CCCCCC)C(=O)OCCCCCCN(CCCCCCOC(=O)C(CCCCCC)CCCCCCCC)CCOC(=O)NCCN(CC)CC] |
| 2 | 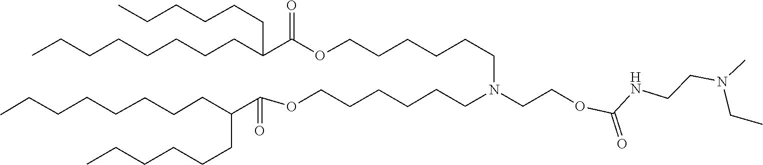 [SMILES: CCCCCCCCC(CCCCCC)C(=O)OCCCCCCN(CCCCCCOC(=O)C(CCCCCC)CCCCCCCC)CCOC(=O)NCCN(C)CC] |
| 3 | 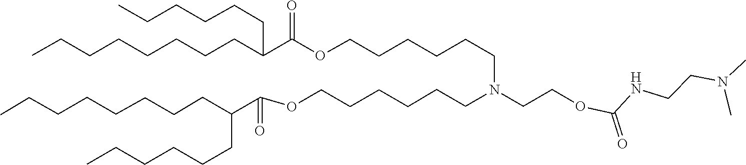 [SMILES: CCCCCCCCC(CCCCCC)C(=O)OCCCCCCN(CCCCCCOC(=O)C(CCCCCC)CCCCCCCC)CCOC(=O)NCCN(C)C] |
| 4 | 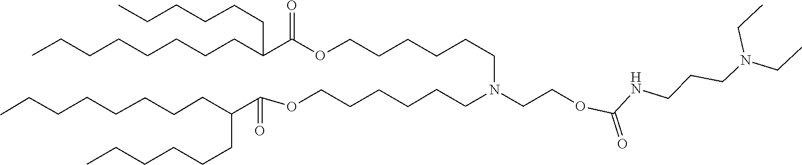 [SMILES: CCCCCCCCC(CCCCCC)C(=O)OCCCCCCN(CCCCCCOC(=O)C(CCCCCC)CCCCCCCC)CCOC(=O)NCCCN(CC)CC] |
| 5 | 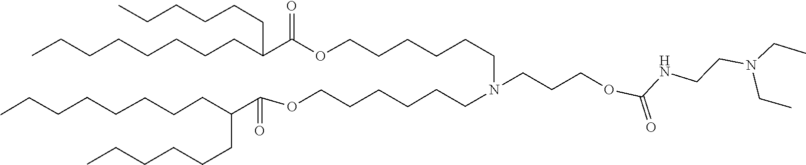 [SMILES: CCCCCCCCC(CCCCCC)C(=O)OCCCCCCN(CCCCCCOC(=O)C(CCCCCC)CCCCCCCC)CCCOC(=O)NCCN(CC)CC] |
| 6 | 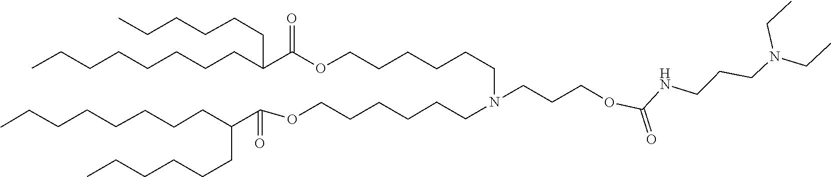 [SMILES: CCCCCCCCC(CCCCCC)C(=O)OCCCCCCN(CCCCCCOC(=O)C(CCCCCC)CCCCCCCC)CCCOC(=O)NCCCN(CC)CC] |
| 7 | 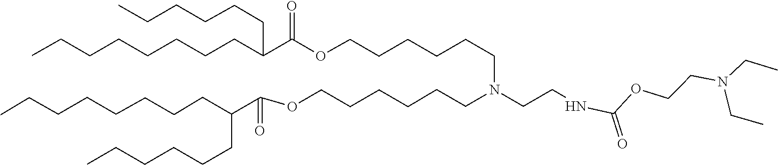 [SMILES: CCCCCCCCC(CCCCCC)C(=O)OCCCCCCN(CCCCCCOC(=O)C(CCCCCC)CCCCCCCC)CCNC(=O)OCCN(CC)CC] |
| 8 | 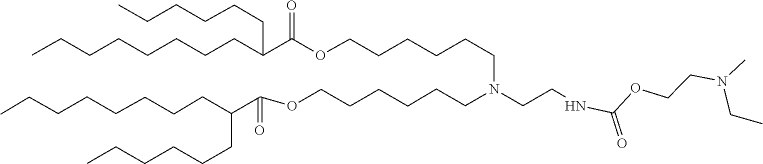 [SMILES: CCCCCCCCC(CCCCCC)C(=O)OCCCCCCN(CCCCCCOC(=O)C(CCCCCC)CCCCCCCC)CCNC(=O)OCCN(C)CC] |
| 9 | 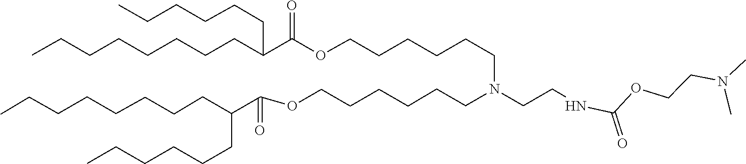 [SMILES: CCCCCCCCC(CCCCCC)C(=O)OCCCCCCN(CCCCCCOC(=O)C(CCCCCC)CCCCCCCC)CCNC(=O)OCCN(C)C] |
| 10 | 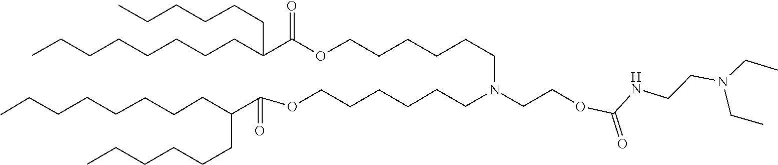 [SMILES: CCCCCCCCC(CCCCCC)C(=O)OCCCCCCN(CCCCCCOC(=O)C(CCCCCC)CCCCCCCC)CCOC(=O)NCCN(CC)CC] |
| 11 | 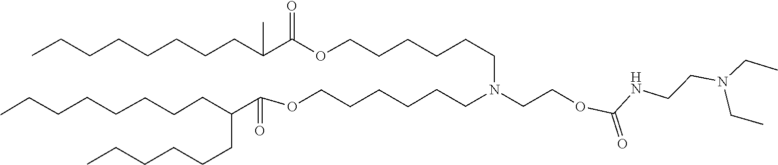 [SMILES: CCCCCCCCC(C)C(=O)OCCCCCCN(CCCCCCOC(=O)C(CCCCCC)CCCCCCCC)CCOC(=O)NCCN(CC)CC] |
| 12 | 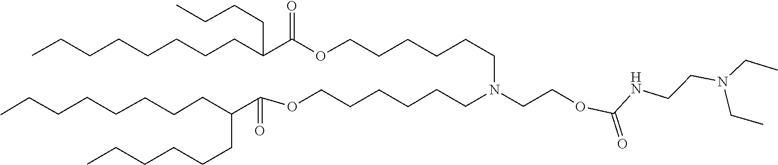 [SMILES: CCCCCCCCC(CCCC)C(=O)OCCCCCCN(CCCCCCOC(=O)C(CCCCCC)CCCCCCCC)CCOC(=O)NCCN(CC)CC] |
| 13 | 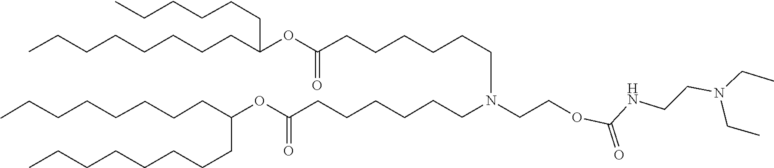 [SMILES: CCCCCCCCC(CCCCCC)OC(=O)CCCCCCN(CCCCCCC(=O)OC(CCCCCCCC)CCCCCCCC)CCOC(=O)NCCN(CC)CC] |
| 14 | 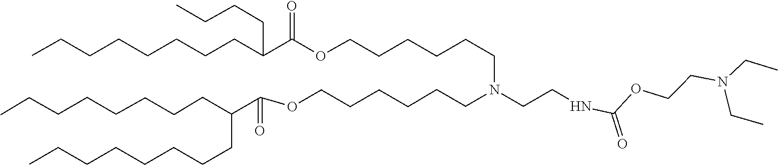 [SMILES: CCCCCCCCC(CCCC)C(=O)OCCCCCCN(CCCCCCOC(=O)C(CCCCCCCC)CCCCCCCC)CCNC(=O)OCCN(CC)CC] |
| 15 | 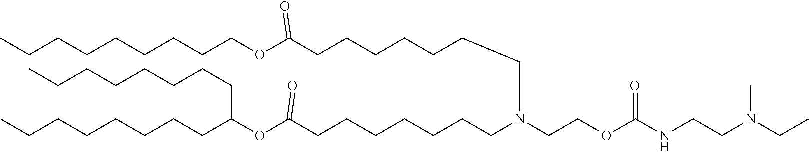 [SMILES: CCCCCCCCCOC(=O)CCCCCCCN(CCCCCCCC(=O)OC(CCCCCCCC)CCCCCCCC)CCOC(=O)NCCN(C)CC] |
| 16 | 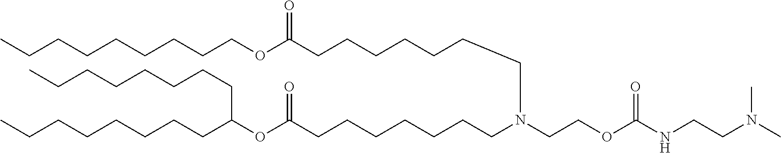 [SMILES: CCCCCCCCCOC(=O)CCCCCCCN(CCCCCCCC(=O)OC(CCCCCCCC)CCCCCCCC)CCOC(=O)NCCN(C)C] |
| 17 | 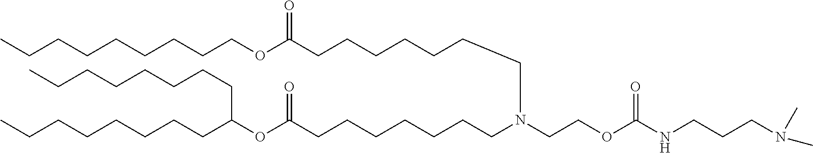 [SMILES: CCCCCCCCCOC(=O)CCCCCCCN(CCCCCCCC(=O)OC(CCCCCCCC)CCCCCCCC)CCOC(=O)NCCCN(C)C] |
| 18 | 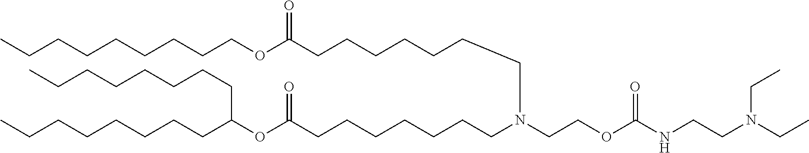 [SMILES: CCCCCCCCCOC(=O)CCCCCCCN(CCCCCCCC(=O)OC(CCCCCCCC)CCCCCCCC)CCOC(=O)NCCN(CC)CC] |
| 19 | 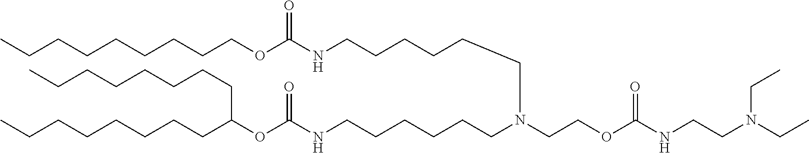 [SMILES: CCCCCCCCCOC(=O)NCCCCCCN(CCCCCCNC(=O)OC(CCCCCCCC)CCCCCCCC)CCOC(=O)NCCN(CC)CC] |
| 20 | 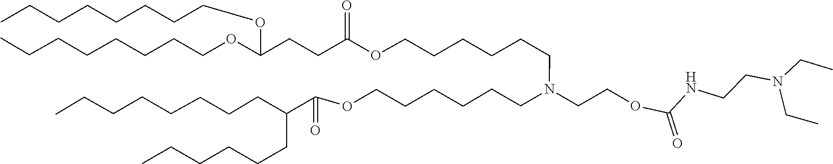 [SMILES: CCCCCCCCOC(CCC(=O)OCCCCCCN(CCCCCCOC(=O)C(CCCCCC)CCCCCCCC)CCOC(=O)NCCN(CC)CC)OCCCCCCCC] |
| 21 | 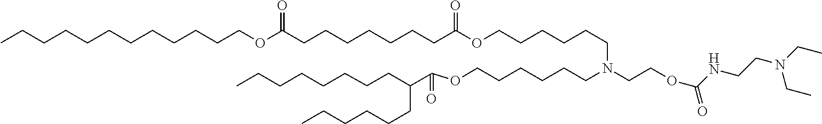 [SMILES: CCCCCCCCCCCCOC(=O)CCCCCCCC(=O)OCCCCCCN(CCCCCCOC(=O)C(CCCCCC)CCCCCCCC)CCOC(=O)NCCN(CC)CC] |
| 22 | 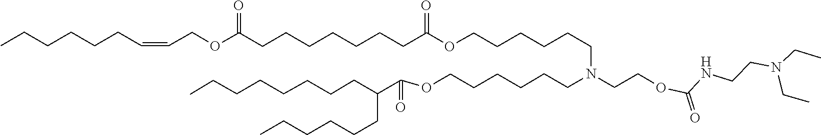 [SMILES: CCCCCC/C=C\COC(=O)CCCCCCCC(=O)OCCCCCCN(CCCCCCOC(=O)C(CCCCCC)CCCCCCCC)CCOC(=O)NCCN(CC)CC] |
| 23 | 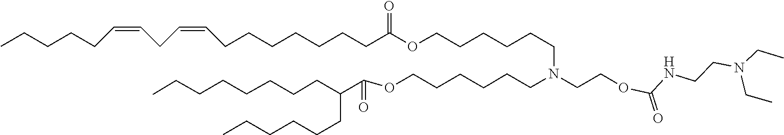 [SMILES: CCCCC/C=C\C/C=C\CCCCCCCC(=O)OCCCCCCN(CCCCCCOC(=O)C(CCCCCC)CCCCCCCC)CCOC(=O)NCCN(CC)CC] |
| 24 | 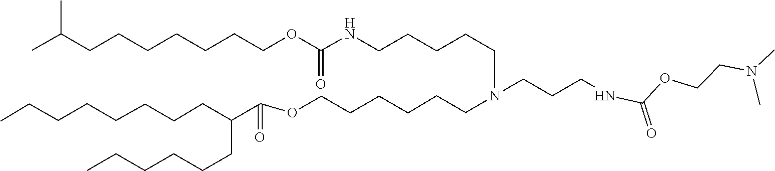 [SMILES: CCCCCCCCC(CCCCCC)C(=O)OCCCCCCN(CCCCCNC(=O)OCCCCCCCC(C)C)CCCNC(=O)OCCN(C)C] |
| 25 | 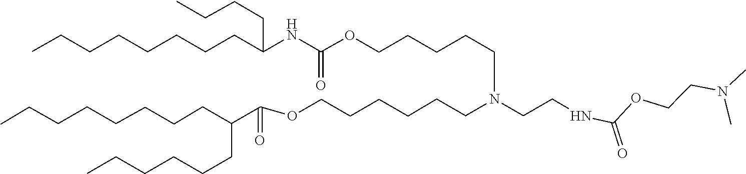 [SMILES: CCCCCCCCC(CCCC)NC(=O)OCCCCCN(CCCCCCOC(=O)C(CCCCCC)CCCCCCCC)CCNC(=O)OCCN(C)C] |
| 26 | 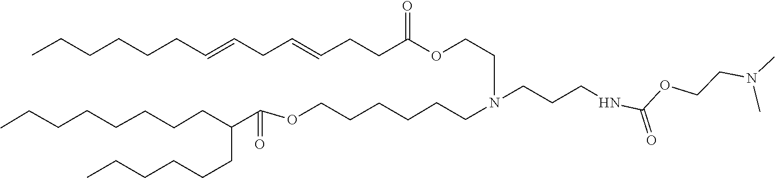 [SMILES: CCCCCC/C=C/C/C=C/CCC(=O)OCCN(CCCCCCOC(=O)C(CCCCCC)CCCCCCCC)CCCNC(=O)OCCN(C)C] |
| 27 | 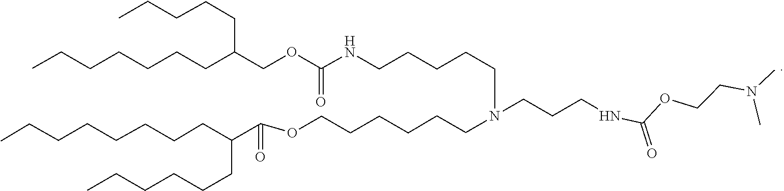 [SMILES: CCCCCCCCC(CCCCCC)C(=O)OCCCCCCN(CCCCCNC(=O)OCC(CCCCC)CCCCCCC)CCCNC(=O)OCCN(C)C] |

In the second aspect, the present disclosure provides a lipid carrier, comprising a compound represented by formula (1) or a pharmaceutically acceptable salt thereof as an ionizable lipid.

In some embodiments, the lipid carrier comprises a compound represented by formula (1) or a pharmaceutically acceptable salt thereof, a helper lipid, a structural lipid, and a polymer-conjugated lipid.

In some embodiments, the helper lipid is at least one selected from 1,2-dioleoyl-sn-glycero-3-phosphatidylethanolamine DOPE, 1,2-distearoyl-sn-glycero-3-phosphatidylcholine DSPC, dioleoylphosphatidylserine DOPS, distearoylphosphatidylserine DSPS, 1,2-distearoyl-sn-glycero-3-phosphoethanolamine DSPE, dipalmitoylphosphatidylserine DPPS, 1,2-dipalmitoyl-sn-glycero-3-phosphatidylcholine DPPC, 1,2-dioleoyl-sn-glycero-3-phosphatidylcholine DOPC, dipalmitoylphosphatidylglycerol DPPG, oleoylphosphatidylcholine POPC, 1-palmitoyl-2-oleoylphosphatidylethanolamine POPE, 1,2-dipalmitoyl-sn-glycero-3-phosphoethanolamine DPPE, 1,2-dimyristoyl-sn-glycero-3-phosphoethanolamine DMPE, distearoyl phosphatidylethanolamine DSPE, and 1-stearoyl-2-oleoylphosphatidylethanolamine SOPE.

In some embodiments, the structural lipid is at least one selected from cholesterol, nonsterol, sitosterol, ergosterol, campesterol, stigmasterol, brassicasterol, tomatine, ursolic acid, α-tocopherol, coprosterol, and corticosteroid.

In some embodiments, the polymer-conjugated lipid is at least one selected from 1,2-dimyristoyl-rac-glycero-3-methoxypolyethylene glycol 2000 (DMG-PEG2000), DMG-PEG2000-mannose, cholesterol-PEG2000, 1,2-dimyristoyl-sn-glycerol methoxy-polyethylene glycol PEG-DMG, dimyristoylglycerol-polyethylene glycol PEG-c-DMG, polyethylene glycol-dimyristoylglycerol PEG-C14, PEG-1,2-dimyristoyloxypropyl-3-amine PEG-c-DMA, 1,2-distearoyl-sn-glycero-3-phosphoethanolamine-N-[amino(polyethylene glycol)] PEG-DSPE, PEGylated phosphatidylethanolamine PEG-PE, PEG-modified ceramide, PEG-modified dialkylamine, PEG-modified diacylglycerol, Tween-20, Tween-80, 1,2-dipalmityl-sn-glycero-methoxypolyethylene glycol PEG-DPG, 4-O-(2′,3′-di(tetradecanoyloxy)propyl-1-O-(ω-methoxy(polyethoxy)ethyl)succinate PEG-s-DMG, PEG-dialkoxypropyl PEG-DAA, mPEG2000-1,2-di-O-alkyl-sn3-carbamoylglyceride PEG-c-DOMG, and N-acetylgalactosamine((R)-2,3-bis(octadecyloxy)propyl-1-(methoxypoly(ethylene glycol)2000)propylcarbamate)) GalNAc-PEG-DSG.

In some embodiments, in the lipid carrier, the molar ratio of the ionizable lipid, the helper lipid, the structural lipid, and the polymer-conjugated lipid is (20 to 75):(2 to 25):(15 to 55):(0 to 15); exemplarily, the molar ratio of the ionizable lipid, the helper lipid, the structural lipid, and the polymer-conjugated lipid may be 45:10:42:3, 30:25:30:10, 46:15:40:3, 50:10:38.5:1.5, 50:10:37:3, 50:9:38:3, etc.

In the third aspect, the present disclosure provides a nucleic acid lipid nanoparticle composition, comprising a compound represented by formula (1) or a pharmaceutically acceptable salt thereof or the aforesaid lipid carrier, and a nucleic acid.

In some embodiments, the nucleic acid is at least one selected from DNA, RNA, a complex containing DNA or RNA (e.g., a complex of DNA and RNA, a complex of DNA and polypeptide/protein, and a complex of RNA and polypeptide/protein), a modified DNA, a modified RNA, and a modified complex containing DNA or RNA.

In some embodiments, the RNA is mRNA, siRNA, dsRNA, rRNA, circRNA, saRNA, tRNA, snRNA or shRNA, preferably mRNA.

In some embodiments, the mass ratio of the aforesaid nucleic acid to any one of the aforesaid compounds or pharmaceutically acceptable salt thereof is 1:(3 to 40).

In some embodiments, the mass ratio of the aforesaid nucleic acid to the aforesaid lipid carrier is 1:(3 to 40).

Exemplarily, the above mass ratio is 1:3, 1:5, 1:10, 1:15, 1:20, 1:30, etc.

In the fourth aspect, the present disclosure provides a pharmaceutical preparation, comprising any one of the aforesaid compounds or pharmaceutically acceptable salts thereof, or the aforesaid lipid carrier, or the aforesaid nucleic acid lipid nanoparticle composition, and a pharmaceutically acceptable carrier.

In some embodiments, the pharmaceutical preparation has a particle size of 30 to 500 nm; exemplarily, the particle size may be 30 nm, 50 nm, 60 nm, 80 nm, 100 nm, 120 nm, 150 nm, 250 nm, 350 nm, 500 nm, etc.

In some embodiments, the encapsulation efficiency of nucleic acids in the pharmaceutical preparation is greater than 50%. Exemplarily, the encapsulation efficiency may be 55%, 60%, 65%, 70%, 75%, 79%, 80%, 85%, 89%, 90%, 93%, 95%, 97%, etc.

In the fifth aspect, the present disclosure also provides use of the aforesaid compound or pharmaceutically acceptable salt thereof, or the aforesaid lipid carrier, or the aforesaid nucleic acid lipid nanoparticle composition, or the aforesaid pharmaceutical preparation in preparation of a nucleic acid drug or a gene vaccine.

The present disclosure further provides a method for in vivo delivery of a nucleic acid drug or a gene vaccine, the method comprising administering, to a subject in need thereof, the aforesaid nucleic acid lipid nanoparticle composition or the aforesaid pharmaceutical preparation.

In some embodiments, the aforesaid nucleic acid lipid nanoparticle composition or the aforesaid pharmaceutical preparation is administered via one of the following routes of administration: oral, intranasal, intravenous, intraperitoneal, intramuscular, intraarticular, intralesional, intratracheal, subcutaneous, and intradermal. In some embodiments, the aforesaid nucleic acid lipid nanoparticle composition or the aforesaid pharmaceutical preparation is administered, for example, via enteral or parenteral administration. In some embodiments, the nucleic acid lipid nanoparticle composition or the pharmaceutical preparation is administered to the subject at a dose of about 0.001 mg/kg to about 10 mg/kg.

### BRIEF DESCRIPTION OF THE DRAWINGS

FIG. 1: Proton nuclear magnetic resonance spectrum of Compound 1.

FIG. 2: Proton nuclear magnetic resonance spectrum of Compound 11.

FIG. 3: Proton nuclear magnetic resonance spectrum of Compound 12.

FIG. 4: Proton nuclear magnetic resonance spectrum of Compound 15.

FIG. 5: Proton nuclear magnetic resonance spectrum of Compound 16.

FIG. 6: Proton nuclear magnetic resonance spectrum of Compound 17.

FIG. 7: Proton nuclear magnetic resonance spectrum of Compound 18.

FIG. 8: Proton nuclear magnetic resonance spectrum of Compound 2.

### DETAILED DESCRIPTION

To render the present disclosure more understandable, some technical and scientific terms are specifically defined below. Unless otherwise explicitly defined herein, all of the other technical and scientific terms used herein have the same meanings as typically understood by one of ordinary skill in the art to which the present disclosure belongs.

In the present specification, the numerical range represented by “numerical value A to numerical value B” refers to the range including the endpoint values A and B. When the upper limit and lower limit of a numerical range are disclosed, any value or any sub-range within this range means to be specifically disclosed. In particular, each of the numerical ranges (e.g., in the form of “from about a to b”, or equivalently “from approximately a to b”, or equivalently “about α-b”) of the parameters disclosed herein should be construed to encompass each of the values and sub-ranges therein. For example, “C₁₋₄” should be construed to encompass any sub-range and each point value therein, e.g., C₂₋₄, C₃₋₄, C₁₋₂, C₁₋₃ or C₁₋₄, and C₁, C₂, C₃ or C₄.

The term “including”, “comprising”, “having” or “containing” or any other variations thereof is intended to encompass non-exclusive or open-ended inclusions. For example, a composition, method or apparatus including a series of elements is not necessarily limited only to the elements that have been explicitly listed, and may also include other elements that are not explicitly listed or elements innate in the above composition, method or apparatus.

In the present specification, the term “optional” or “optionally” means that the event or case described subsequently may or may not occur, and the description includes the case where the event occurs and the case where the event does not occur.

Phrases such as “some specific/preferred embodiments”, “other specific/preferred embodiments”, and “embodiments” referred to in the present specification mean that particular elements (for example, features, structures, properties and/or characteristics) described in relation to this embodiment are included in at least one of the embodiments described herein, and may or may not exist in other embodiments. Additionally, it should be appreciated that the elements may be combined in any suitable manner into various embodiments.

Before the present disclosure is further described, it shall be understood that the present disclosure is not limited to the particular embodiments described herein. It should also be understood that the terms used herein are intended only to describe rather than limit the particular embodiments.

The term “pharmaceutically acceptable salt” refers to salts of the compounds of the present disclosure that are substantially non-toxic to an organism. Pharmaceutically acceptable salts generally include (but are not limited to) salts formed by reacting the compounds of the present disclosure with a pharmaceutically acceptable inorganic/organic acid or inorganic/organic base, and such salts are also referred to as acid addition salts or base addition salts. Common inorganic acids include (but are not limited to) hydrochloric acid, hydrobromic acid, sulfuric acid, phosphoric acid, and the like. Common organic acids include (but are not limited to) trifluoroacetic acid, citric acid, maleic acid, fumaric acid, succinic acid, tartaric acid, lactic acid, pyruvic acid, oxalic acid, formic acid, acetic acid, benzoic acid, methanesulfonic acid, benzenesulfonic acid, p-toluenesulfonic acid, and the like. Common inorganic bases include (but are not limited to) sodium hydroxide, potassium hydroxide, calcium hydroxide, barium hydroxide, and the like. Common organic bases include (but are not limited to) diethylamine, triethylamine, ethambutol, and the like.

The term “pharmaceutically acceptable carrier” refers to an excipient administered together with the aforesaid nucleic acid lipid nanoparticle composition or the aforesaid pharmaceutical preparation, which is suitable, within the scope of reasonable medical judgment, for contact with the tissue of human beings and/or other animals without undue toxicity, irritation, allergic response or other problems or complications commensurate with a reasonable benefit/risk ratio. Pharmaceutically acceptable carriers usable in the present disclosure include, but are not limited to, a) diluents; b) lubricants; c) binders; d) disintegrants; e) absorbents, colorants, flavoring agents, and/or sweetening agents; f) emulsifiers or dispersants; and/or g) substances that enhance absorption of the compounds.

The term “independently” means that at least two groups (or ring systems) with the same or similar range in the structure may have the same or different meanings under particular circumstances. For example, if the substituent X and the substituent Y are each independently hydrogen, halogen, hydroxyl, cyano, alkyl, or aryl, when the substituent X is hydrogen, the substituent Y may either be hydrogen or be halogen, hydroxyl, cyano, alkyl, or aryl; similarly, when the substituent Y is hydrogen, the substituent X may either be hydrogen or be halogen, hydroxyl, cyano, alkyl, or aryl.

The term “alkyl” refers to monovalent, linear or branched, saturated aliphatic hydrocarbyl. For example, “C₁₋₃₀ alkyl” refers to monovalent, linear or branched, saturated aliphatic hydrocarbyl including 1 to 30 carbon atoms, e.g., alkyl may be methyl, ethyl, n-propyl, isopropyl, n-butyl, sec-butyl, isobutyl, or tert-butyl.

The term “alkylene” refers to divalent, linear or branched, saturated aliphatic hydrocarbyl, in which the two groups (or fragments) it links may be linked either to the same carbon atom or to different carbon atoms. For example, the term “C₁₋₄ alkylene” used herein refers to alkylene having 1 to 4 carbon atoms (such as methylene, 1,1-ethylene, 1,2-ethylene, 1,2-propylene, or 1,3-butylene).

The term “alkenyl” refers to linear or branched, unsaturated aliphatic hydrocarbyl consisting of carbon atoms and hydrogen atoms and having at least one double bond. For example, “C₂₋₃₀ alkenyl” refers to monovalent, linear or branched, hydrocarbyl containing 2 to 30 carbon atoms and having at least one carbon-carbon double bond. Non-limiting examples of alkenyl include, but are not limited to, vinyl, 1-propenyl, 2-propenyl, 1-butenyl, isobutenyl, 1,3-butadienyl, and the like.

In order to render the purposes and technical solutions of the present disclosure clearer, the embodiments of the present disclosure will be described in detail below with reference to the examples. A person skilled in the art will appreciate, however, that the following examples are intended only to illustrate the present disclosure and shall not be considered to limit the scope of the present disclosure.

The reagents or instruments used in the examples are all commercially-available conventional products. Where the specific conditions are not specified, conventional conditions or those recommended by the manufacturers are followed. The term “room temperature” used herein refers to 20° C.±5° C. When used to modify a value or a numerical range, the term “about” used herein is meant to include this value or numerical range and an error range of this value or numerical range acceptable to a person skilled in the art, for example, the error range is ±10%, ±5%, ±4%, ±3%, ±2%, ±1%, or 0.5%.

In the following examples, all the experimental methods employed are conventional methods, unless otherwise specified; and all the reagents and materials are commercially available, unless otherwise specified.

In the following examples, all the solvents and drugs used are analytically or chemically pure; all the solvents have been re-distilled before use; and all the anhydrous solvents are treated according to standard methods or literature methods.

The abbreviations used herein have the following meanings:

| Abbrev. | Meaning | Abbrev. | Meaning |
|---|---|---|---|
| DCM | Dichloromethane | EDCI | 1-Ethyl-(3-dimethylaminopropyl)carbodiimide |
| DMAP | 4-Dimethylaminopyridine | ACN | Acetonitrile |
| DIEA | N,N-Diisopropylethylamine | CDI | N,N′-Carbonyldiimidazole |
| TEA | Triethylamine | HPLC | High Performance Liquid Chromatography |
| DCE | Dichloroethane | | |

The compounds of the present disclosure are separated and purified by preparative TLC, silica gel column chromatography, Prep-HPLC and/or flash column chromatography, and their structures are confirmed by ¹H NMR and/or MS. The reactions are monitored by TLC or LC-MS.

¹H NMR spectroscopy is conducted using a Bruker superconducting nuclear magnetic resonance spectrometer (model: AVACE III HD 400 MHz).

LC/MS is conducted using Aglient 1260 Infinity/Aglient 6120 Quadrupole.

TLC is conducted using silica gel GF 254 as a stationary phase.

Column chromatography is generally conducted using 200- to 300-mesh silica gel (available from Qingdao Haiyang Chemical Co., Ltd.) as a stationary phase.

Flash column chromatography is conducted using Biotage flash column chromatograph.

Prep-HPLC is conducted using Agilent 1260 type and Waters 2489 type.

In the following examples, the reaction temperature is room temperature (15° C. to 30° C.), unless otherwise specified.

### Synthesis Example 1: Preparation Method for Intermediate 1

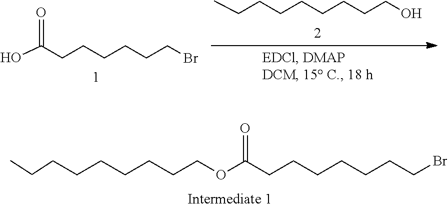

Raw material 1 (20 g, 89.64 mmol, 1 eq) and raw material 2 (26.51 g, 183.77 mmol, 2.05 eq) were dissolved into DCM (400 mL), and EDCI (17.18 g, 89.64 mmol, 1 eq) and DMAP (2.19 g, 17.93 mmol, 0.2 eq) were added, and reacted with stirring at 15° C. for 18 hours. The reaction mixture was diluted with 200 mL of H₂O and extracted with 400 mL of DCM. The organic layers were combined, washed, dried, filtered, and concentrated under vacuum. The crude oily product was purified by flash column chromatography to afford a colorless liquid, which was Intermediate 1 as the target product.

### Synthesis Example 2: Preparation Method for Intermediate 2

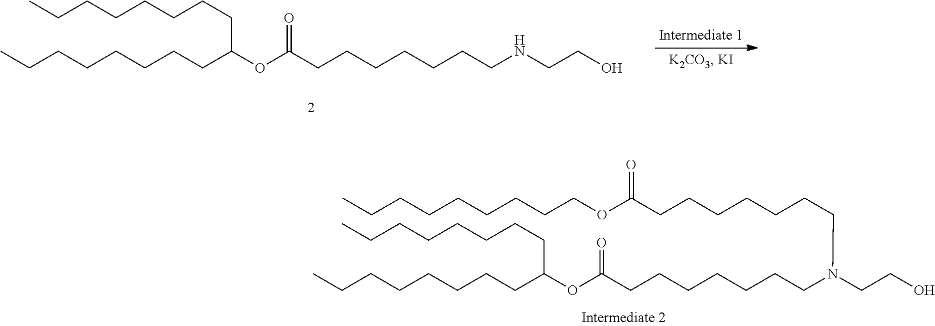 [SMILES: CCCCCCCCC(CCCCCCCC)OC(=O)CCCCCCCNCCO ; CCCCCCCCCOC(=O)CCCCCCCN(CCO)CCCCCCCC(=O)OC(CCCCCCCC)CCCCCCCC]

Raw material 2 (14.2 g, 25.72 mmol, 1 eq) and the Intermediate 1 (8.98 g, 25.72 mmol, 1 eq) prepared in Synthesis Example 1 were dissolved into ACN (250 mL), and K₂CO₃ (14.22 g, 102.87 mmol, 4 eq) and KI (4.70 g, 28.29 mmol, 1.1 eq) were added, diluted with cyclopentyl methyl ether (60 mL), heated at 90° C., and reacted with stirring for 16 hours under N₂. The reaction mixture was filtered. The filtrate was diluted with 100 mL of H₂O and extracted with 200 mL of DCM. The organic layers were combined, washed, dried, filtered, concentrated under vacuum, and purified by flash column chromatography to afford a yellow liquid, which was Intermediate 2.

### Synthesis Example 3: Preparation Method for Raw Material 4

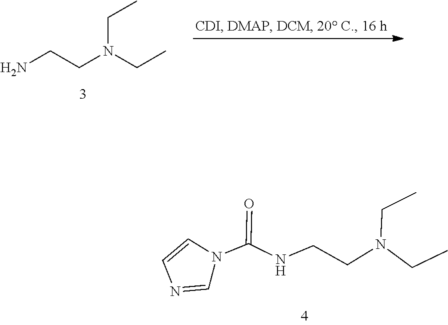

Raw material 3 (300 mg, 422.44 umol, 1 eq) was dissolved into DCM (4 mL), and DIEA (272.99 mg, 2.11 mmol, 367.91 uL, 5 eq) and CDI (205.49 mg, 1.27 mmol, 3 eq) were added, and reacted with stirring at 20° C. for 16 hours. The reaction mixture was diluted with 20 mL of H₂O and extracted with 40 mL of DCM. The organic layers were combined, washed, dried, filtered, and concentrated under reduced pressure to afford a yellow gel-like product, which was the raw material 4.

### Example 1: Preparation Method for Compound 18

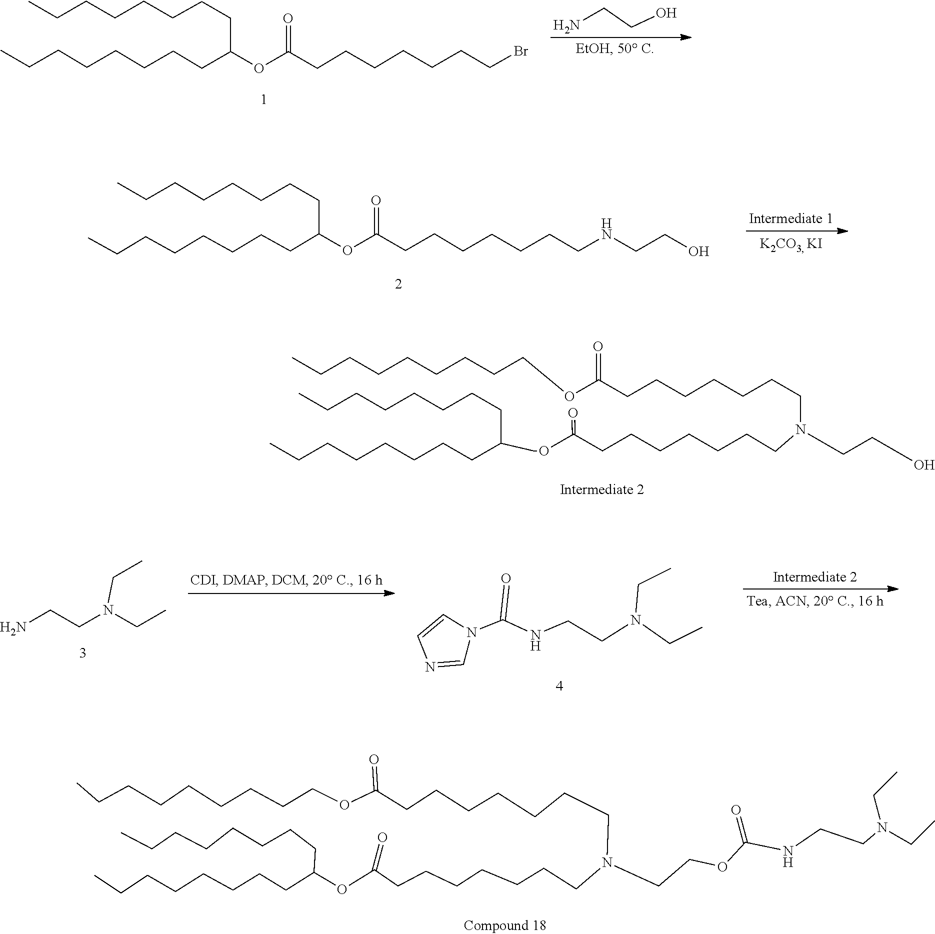

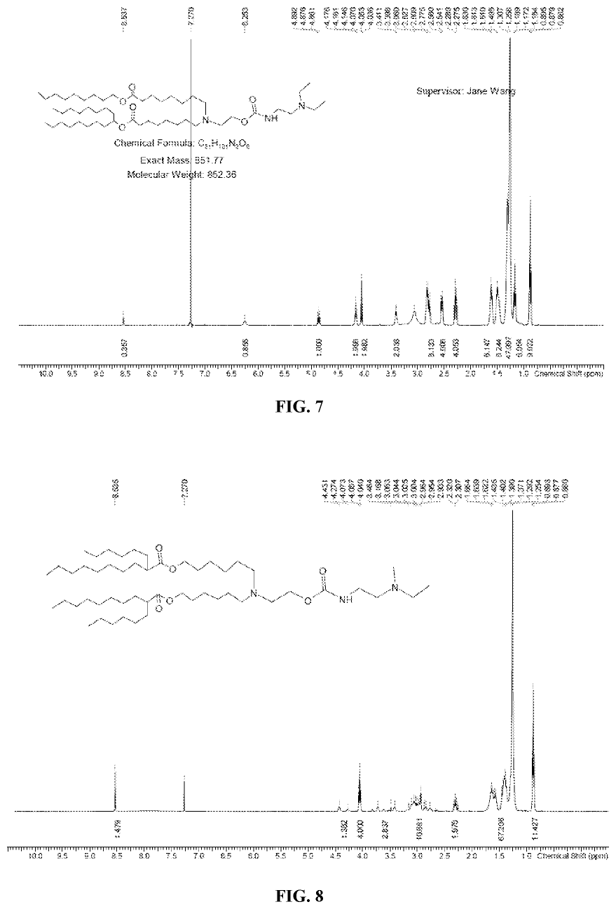

The Intermediate 2 (370 mg) prepared in Synthesis Example 2 was dissolved into ACN (4 mL), and TEA (325.88 mg) and the raw material 4 (187.12 mg) were added, and reacted with stirring at 20° C. for 16 hours. The mixture was concentrated under reduced pressure. The residue was purified by preparative HPLC to afford a red oily product, which was Compound 18 (126.9 mg). The proton nuclear magnetic resonance (¹H NMR) spectral data for Compound 18 were as shown in FIG. 7.

### Example 2: Preparation Method for Compound 15

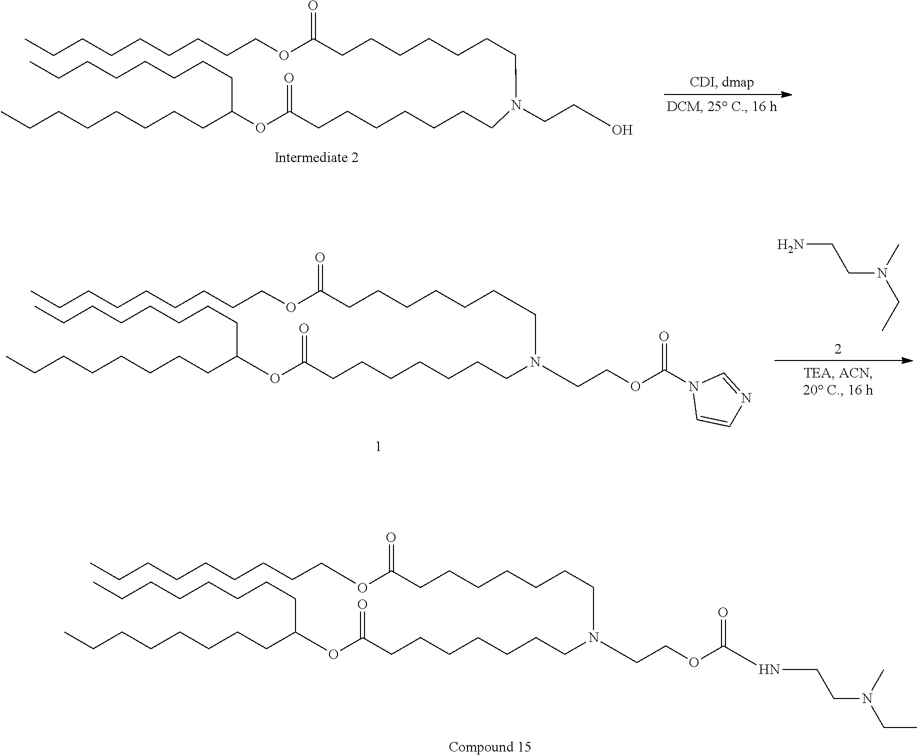

The Intermediate 2 (2 g) was dissolved into DCE (40 mL), and DIEA (1.82 g) and CDI (2.74 g) were added and reacted with stirring at 50° C. for 16 hours. The reaction product was diluted with 50 mL of H₂O and extracted with 100 mL of DCM. The organic layers were combined, washed, dried, filtered, and concentrated under reduced pressure to afford a yellow oily product, which was the raw material 1 (2.49 g).

The raw material 1 (1 g) was dissolved into ACN (5 mL), and TEA (629.11 mg), DMAP (75.95 mg) and the raw material 2 (635.25 mg) were added and reacted with stirring at 50° C. for 16 hours. The mixture was concentrated under reduced pressure. The resulting residue was purified by preparative HPLC to afford a red oily product, which was Compound 15. The ¹H NMR spectral data for Compound 15 were as shown in FIG. 4.

### Example 3: Preparation Method for Compound 16

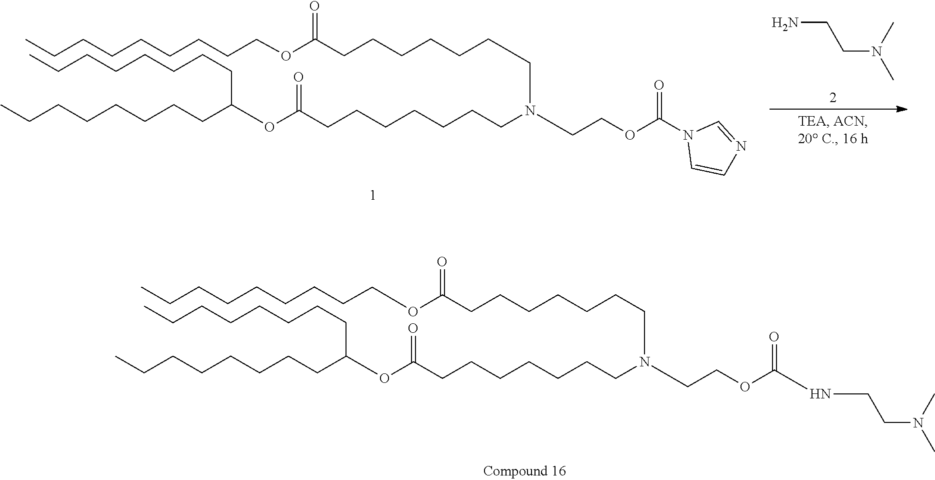

The raw material 1 (3 g) was dissolved into ACN (20 mL), and TEA (1.89 g), DMAP (227.86 mg) and the raw material 2 (1.64 g) were added and reacted with stirring at 50° C. for 16 hours. The mixture was concentrated under reduced pressure. The resulting residue was purified by preparative HPLC to afford a red oily product, which was Compound 16. The ¹H NMR spectral data for Compound 16 were as shown in FIG. 5.

### Example 4: Preparation Method for Compound 17

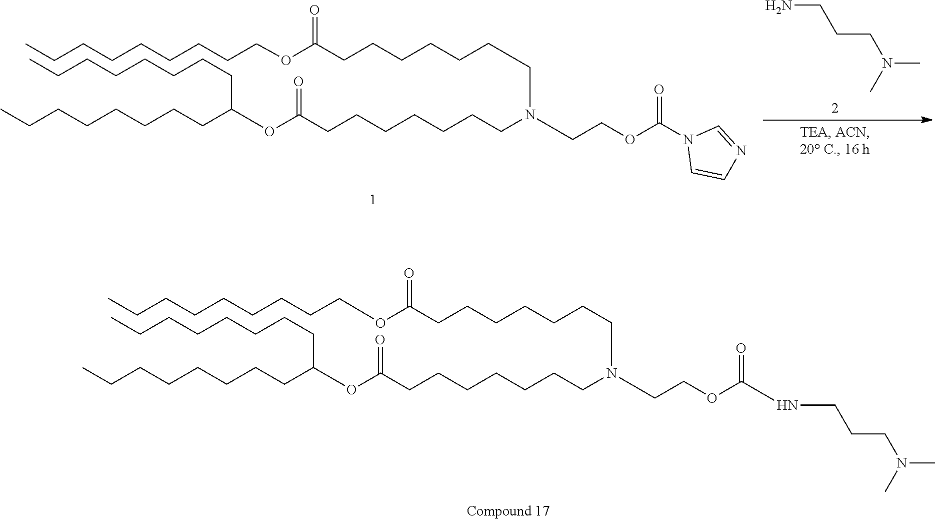

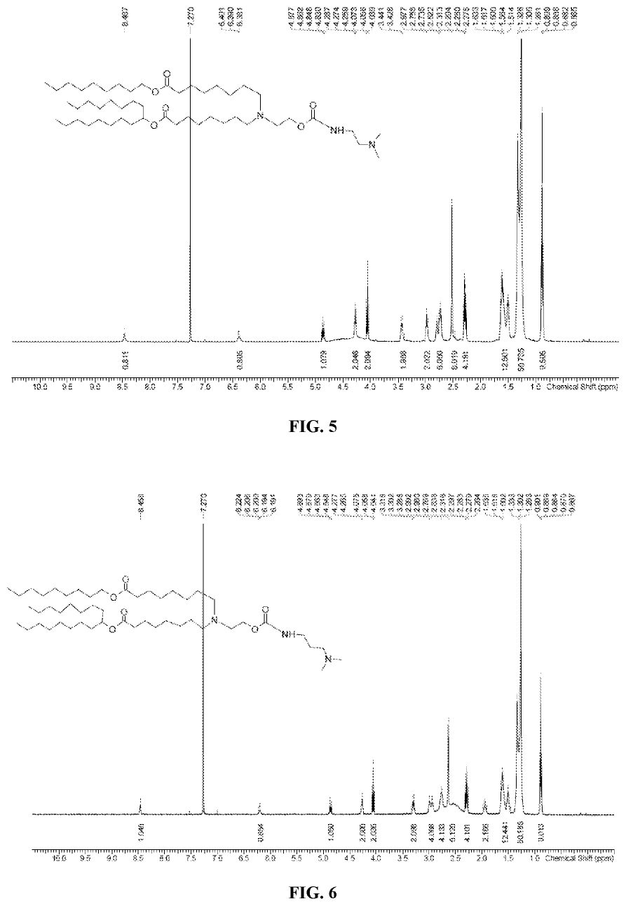

The raw material 1 (3 g) was dissolved into ACN (20 mL), and TEA (1.89 g), DMAP (227.86 mg) and the raw material 2 (1.91 g) were added and reacted with stirring at 50° C. for 16 hours. The mixture was concentrated under reduced pressure. The resulting residue was purified by preparative HPLC to afford a red oily product, which was Compound 17. The ¹H NMR spectral data for Compound 17 were as shown in FIG. 6.

### Example 5: Preparation Method for Compound 1

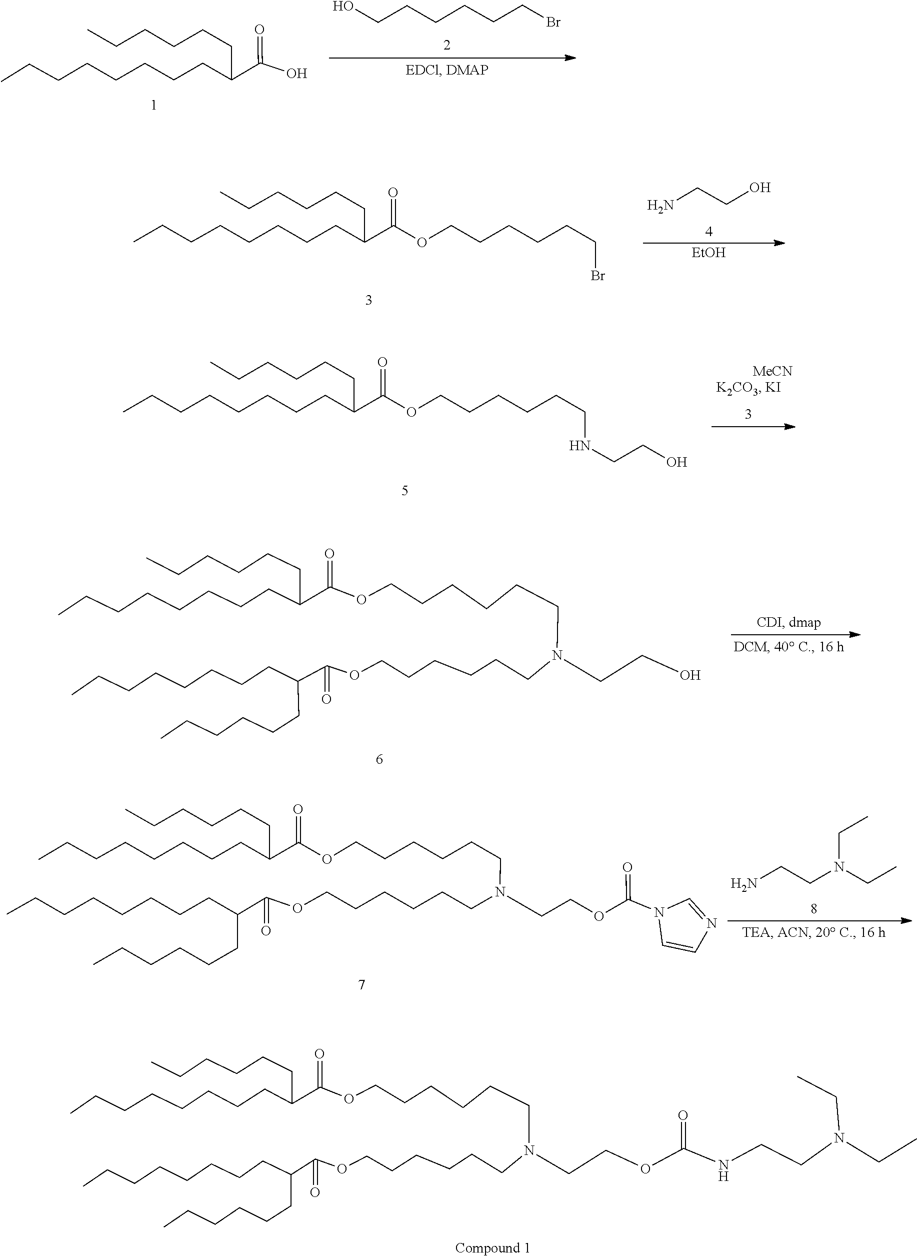

The raw material 1 (5 g) was dissolved into DCM (25 mL), and EDCI (4.49 g), DMAP (2.38 g) and the raw material 2 (3.53 g, 19.50 mmol, 2.56 mL, 1 eq) were added and reacted with stirring at 20° C. for 16 hours. The reaction mixture was concentrated under reduced pressure. The resulting residue was purified by flash silica gel chromatography to afford a colorless oily product, which was raw material 3.

The raw material 3 (2 g) was dissolved into ethanol (1 mL), and raw material 4 (8.74 g) was added and reacted with stirring at 50° C. for 18 hours. The reaction mixture was concentrated under reduced pressure to remove the solvent. The residue was diluted with 80 mL of H₂O and extracted with 200 mL of ethyl acetate. The organic layers were combined, washed, dried, filtered, and concentrated under reduced pressure to afford a yellow liquid, which was the raw material 5.

The raw material 5 (890 mg) and the raw material 3 (1.12 g) were dissolved into ACN (4 mL), and K₂CO₃ (1.23 g) and KI (406.65 mg) were added, diluted with cyclopentyl methyl ether (1 mL), heated at 90° C., and reacted with stirring for 16 hours under N₂. The reaction mixture was concentrated under reduced pressure to remove the solvent. The residue was diluted with 80 mL of H₂O and extracted with 200 mL of ethyl acetate. The organic layers were washed with 100 mL of brine, dried, filtered, and concentrated under reduced pressure. The resulting residue was purified by flash silica gel chromatography to afford a colorless liquid, which was the raw material 6.

To a solution of the raw material 6 (900 mg) in DCE (20 mL), DIEA (1.34 g) and CDI (1.01 g) were added and reacted with stirring at 50° C. for 16 hours. The reaction mixture was concentrated under reduced pressure to remove the solvent. The residue was diluted with 30 mL of H₂O and extracted with 30 mL of dichloromethane. The organic layers were washed with 30 mL of brine, dried, filtered, and concentrated under reduced pressure to afford a yellow oily product, which was the raw material 7.

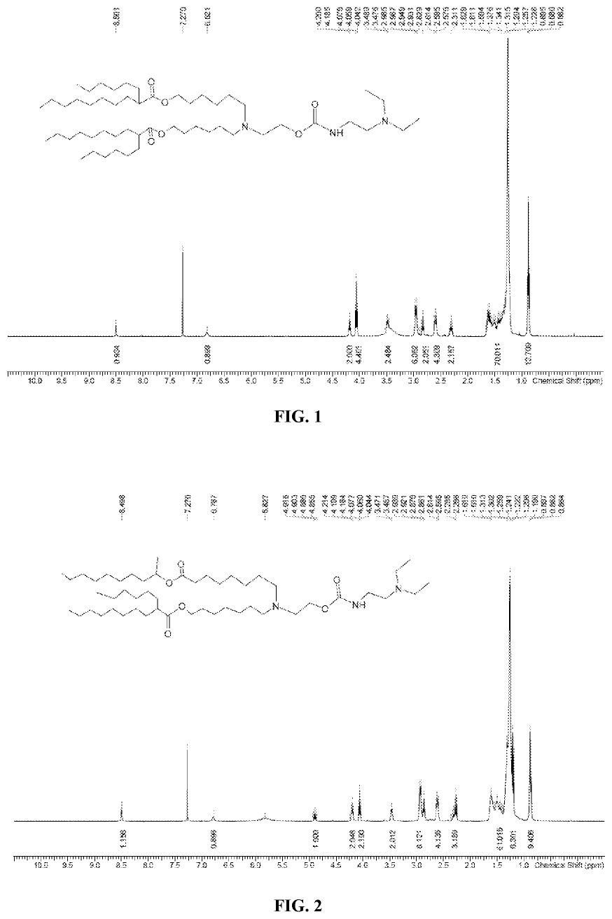

The raw material 7 (600 mg) was dissolved into ACN (5 mL), and TEA (510.64 mg), DMAP (61.65 mg) and raw material 8 (586.41 mg) were added and reacted with stirring at 50° C. for 16 hours. The mixture was concentrated under reduced pressure. The resulting residue was purified by preparative HPLC to afford a red oily product, which was Compound 1. The ¹H NMR spectral data for Compound 1 were as shown in FIG. 1.

### Example 6: Preparation Method for Compound 12

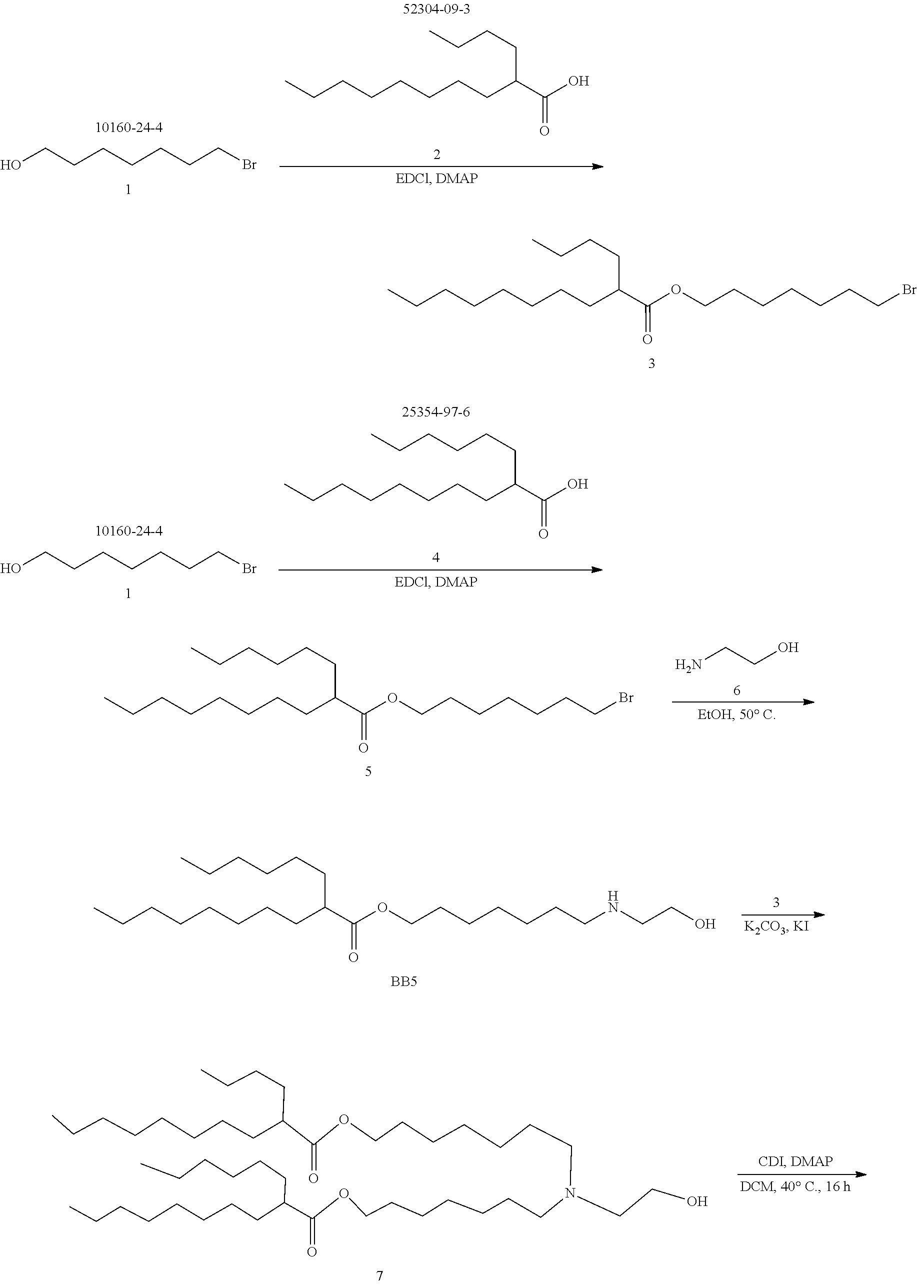

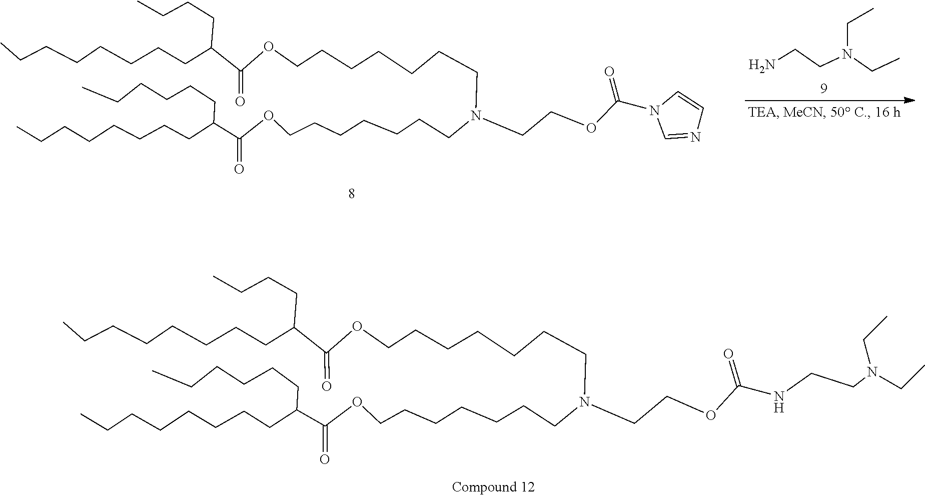

The raw material 2 (1 g) and the raw material 1 (1.28 g) were dissolved into DCM (20 mL), and EDCI (839.43 mg) and DMAP (106.99 mg) were added and reacted with stirring at 15° C. for 18 h. The reaction mixture was diluted with 100 mL of H₂O and extracted with 200 mL of DCM. The organic layers were washed with 100 mL of brine, dried, filtered, and concentrated under reduced pressure. The resulting residue was purified by flash silica gel chromatography to afford a colorless liquid, which was the raw material 3.

The raw material 4 (5 g) and the raw material 1 (5.71 g) were dissolved into DCM (100 mL), and EDCI (3.74 g) and DMAP (476.44 mg) were added and reacted with stirring at 15° C. for 18 hours. The reaction mixture was diluted with 100 mL of H₂O and extracted with 200 mL of DCM. The organic layers were washed with 100 mL of brine, dried, filtered, and concentrated under reduced pressure. The resulting residue was purified by flash silica gel chromatography to afford a colorless liquid, which was the raw material 5.

The raw material 5 (7.06 g) was dissolved into ethanol (6 mL), and the raw material 6 (29.84 g) was added and reacted with stirring at 50° C. for 18 hours. The reaction mixture was diluted with 200 mL of H₂O and extracted with 300 mL (150 mL*2) of ethyl acetate. The organic layers were combined, washed with 300 mL of brine, dried, filtered, and concentrated under reduced pressure to afford a colorless liquid, which was the raw material BB5.

The raw material BB5 (1 g) and the raw material 3 (980.12 mg) were dissolved into cyclopentyl methyl ether (10 mL), and K₂CO₃ (1.34 g) and KI (441.41 mg) were added. The mixture was diluted with ACN (40 mL). The resulting mixture was heated at 90° C. and stirred for 28 hours under N₂. The reaction mixture was cooled at room temperature, filtered, and concentrated under reduced pressure. The resulting residue was purified by flash silica gel chromatography to afford a yellow oily product, which was the raw material 7.

The raw material 7 (800 mg) was dissolved into DCM (8 mL), and DIEA (630.26 mg) and CDI (474.44 mg) were added and reacted with stirring at 50° C. for 16 hours. The reaction mixture was diluted with 15 mL of H₂O and extracted with 30 mL of DCM. The organic layers were washed with 15 mL of brine, dried, filtered, and concentrated under reduced pressure to afford a colorless oily product, which was the raw material 8.

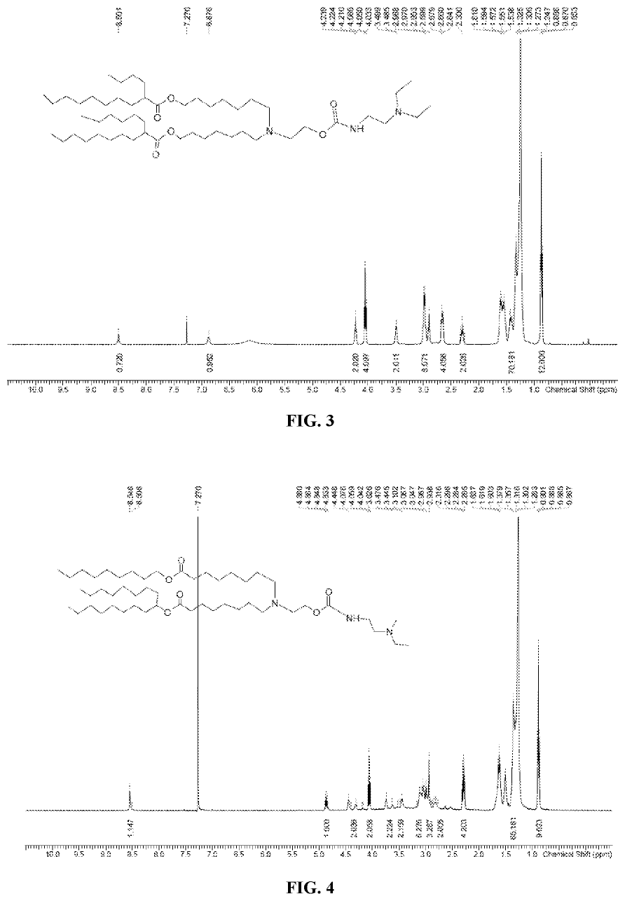

The raw material 8 (1.16 g) was dissolved into ACN (10 mL), and TEA (705.17 mg), DMAP (85.14 mg) and raw material 9 (809.80 mg) were added and stirred at 50° C. for 16 hours. The mixture was concentrated under reduced pressure. The residue was purified by preparative HPLC to afford a yellow oily product, which was Compound 12. The ¹H NMR spectral data for Compound 12 were as shown in FIG. 3.

### Example 7: Preparation Method for Compound 11

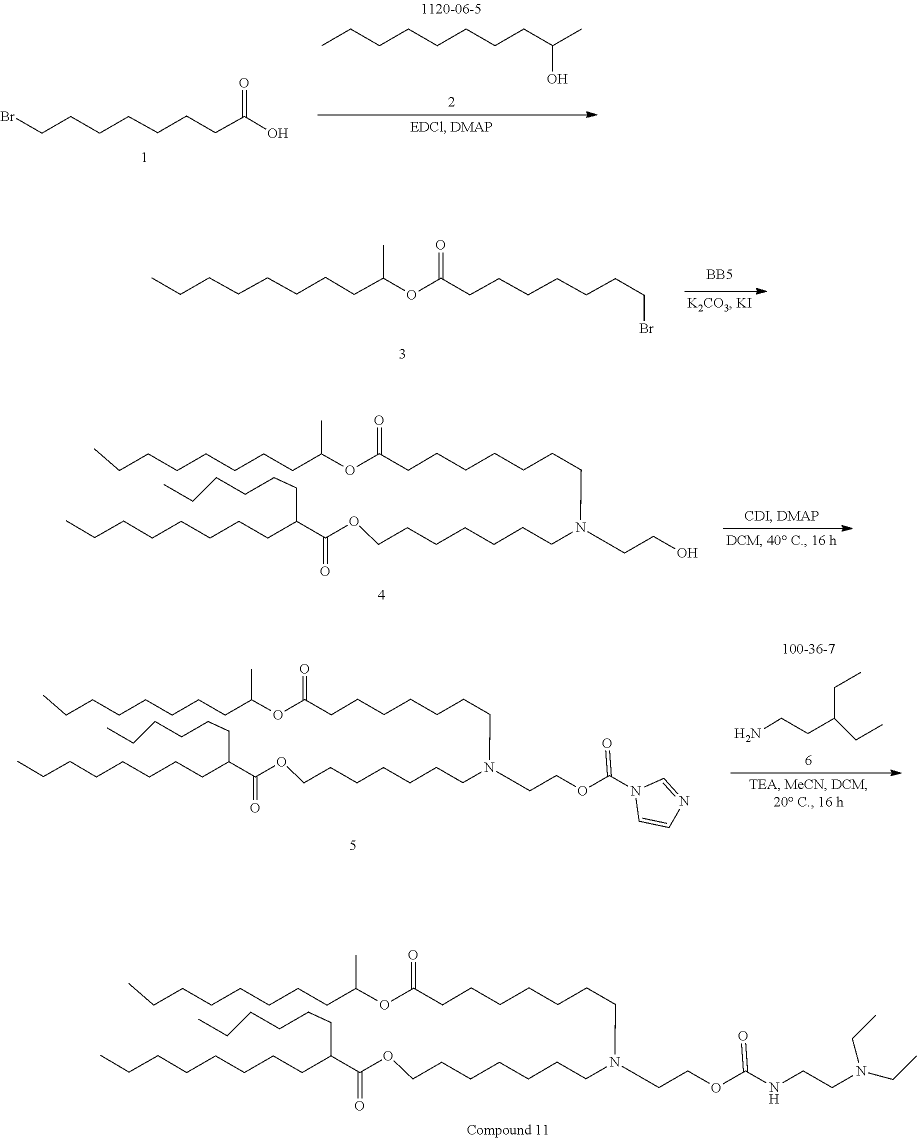

The raw material 1 (4.23 g) and the raw material 2 (2 g) were dissolved into DCM (50 mL), and EDCI (2.42 g) and DMAP (308.74 mg) were added and reacted with stirring at 15° C. for 18 hours. The reaction mixture was diluted with 60 mL of H₂O and extracted with 100 mL of DCM. The organic layers were washed, dried, filtered, and concentrated under reduced pressure. The resulting residue was purified by flash silica gel chromatography to afford a colorless liquid, which was the raw material 3.

The raw material 3 (1 g) and the raw material BB5 (878.40 mg) obtained in Example 6 were dissolved into cyclopentyl methyl ether (10 mL), and K₂CO₃ (1.34 g) and KI (441.41 mg) were added, diluted with ACN (40 mL), heated at 90° C., and reacted with stirring for 28 hours under N₂. The reaction mixture was cooled at room temperature, filtered, and concentrated under reduced pressure. The resulting residue was purified by flash silica gel chromatography to afford a yellow oily product, which was the raw material 4.

The raw material 4 (700 mg) was dissolved into DCM (10 mL), and DIEA (584.81 mg) and CDI (440.23 mg) were added and reacted with stirring at 50° C. for 16 hours. The reaction mixture was diluted with 50 mL of H₂O and extracted with 120 mL of DCM. The organic layers were combined, washed, dried, filtered, and concentrated under reduced pressure to afford a colorless oily product, which was the raw material 5.

The raw material 5 (950 mg) was dissolved into ACN (10 mL), and TEA (608.26 mg), DMAP (73.44 mg) and raw material 6 (698.51 mg) were added and reacted with stirring at 50° C. for 16 hours. The mixture was concentrated under reduced pressure. The resulting residue was purified by preparative HPLC to afford a yellow oily product, which was Compound 11. The ¹H NMR spectral data for Compound 11 were as shown in FIG. 2.

### Example 8: Preparation Method for Compound 2

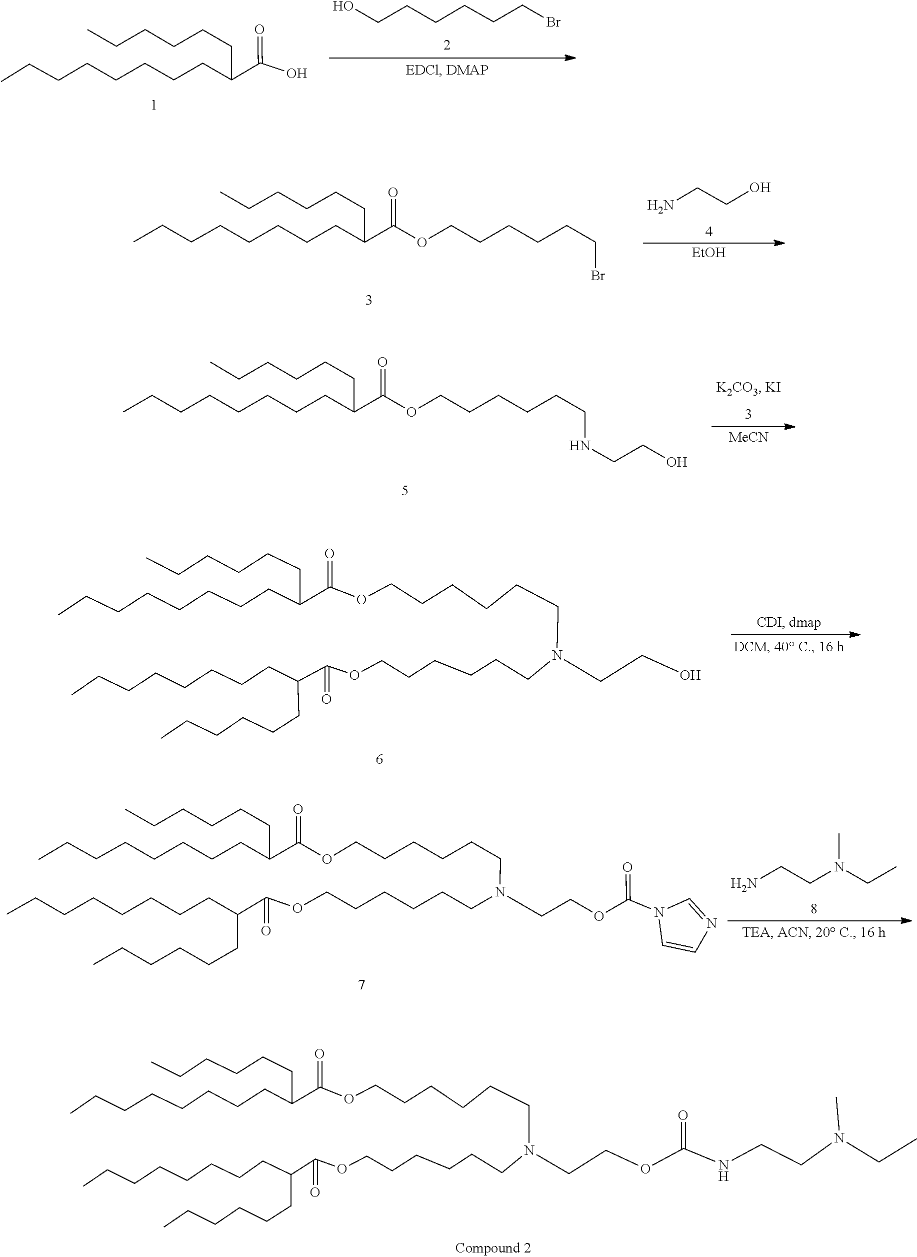

Raw material 7 was obtained in the same way as in Example 5. The raw material 7 (600 mg) was dissolved into ACN (7.9 mL), and TEA (510.64 mg), DMAP (61.6 mg) and raw material 8 (515 mg) were added and reacted with stirring at 50° C. for 16 hours. The mixture was concentrated under reduced pressure. The resulting residue was purified by preparative HPLC to afford a red oily product, which was Compound 2. The ¹H NMR spectral data for Compound 2 were as shown in FIG. 8.

### Example 9: Preparation and Characterization of Lipid Nanoparticles

### 1. Preparation of Lipid Nanoparticles (LNPs) (by Micro & Nano Instrument)

The preparation of lipid nanoparticles involved mixing the above-mentioned ionizable lipid with a helper lipid (such as DSPC, DOPE, and DOPC), a polymer-conjugated lipid (such as DMG-PEG2000), a structural lipid (such as cholesterol and steroid) and the like at a certain molar ratio, and dissolving the mixture into anhydrous ethanol, so that the total lipid phase concentration was 0.5 to 20 mg/mL. Nucleic acids were dissolved into a buffer solution having a pH of less than 7 (e.g., 3 to 6.5), and rapidly mixed with a solution of lipids in ethanol to achieve the preparation purpose.

In this example, the total lipid phase concentration of 1 mg/mL was formulated in anhydrous ethanol at the molar ratio of ionizable lipid (Compound 1, Compound 11 or Compound 12):helper lipid (DSPC or DOPE):cholesterol:DMG-PEG2000=50%:10%:38.5%:1.5%. Luc-eGFP mRNA at the corresponding concentration was dissolved in 50 mM citric acid buffer with an aqueous buffer solution of pH4.0. The mass ratio of mRNA to the total lipids was 1:30.

LNPs were prepared using the formulation-screening chip from Micro & Nano at a total flow rate of 12 ml/min and a flow rate ratio of 1:3 between the lipid phase and the aqueous phase. The lipid nanoparticles were collected and dialyzed with PBS overnight at 4° C. to remove ethanol and acidic buffer salts. The dialysate volume was 400 times or more the sample volume. The dialyzed sample was filtered through a 0.2-μm filter membrane. Lipid nanoparticles could also be prepared by other methods, such as injection mixing, in addition to using microfluidic chips. After being dialyzed and filtered, the sample was concentrated using a 10 kDa cellulose ultrafiltration membrane. The centrifugation parameter was 1000 g to 2000 g, and the centrifugation time was 30 min to 1 hour. The centrifugation temperature was 4° C.

2. Determination of particle size: The particle size and polydispersity index (PDI) of the prepared lipid nanoparticles were measured using a Malvern particle size analyzer.

3. Determination of Encapsulation Efficiency of mRNAs:

For mRNA-encapsulated lipid nanoparticles, the assay specified in the Quant-iT™ RiboGreen® RNA kit could be employed. The specific operations were as follows: The sample was diluted 10 to 20 folds with a TE buffer solution (10 mM Tris-HCl, 1 mM EDTA, pH 7.5). An equal volume of demulsifier (2% Triton X-100) was added to the sample to be tested and then the solution was diluted 10 to 20 folds again. The RiboGreen® reagent was diluted 100 folds in the TE buffer solution, and 100 μl of the diluted solution was added to a 96-well plate. 100 μl of the sample to be tested was added to the corresponding 96-well plate. Meanwhile, a standard curve was plotted using the mRNA standard. The fluorescence value at 520 nm excited by 480 nm excitation light was read using a microplate reader. The concentrations of the encapsulated and free mRNAs were calculated based on the standard curve.

TABLE 1
Particle Sizes and Nucleic Acid Encapsulation
Efficiencies of Lipid Nanoparticles Containing
Different Ionizable Lipids and Helper Lipids

| | | Particle | | Nucleic Acid |
|---|---|---|---|---|
| Ionizable | Helper | Size | | Encapsulation |
| Lipid | Lipid | nm | PDI | Efficiency (%) |
| Compound 1 | DSPC | 111.7 | 0.135 | 93.01 |
| Compound 1 | DOPE | 99.86 | 0.082 | 95.42 |
| Compound 11 | DSPC | 96.88 | 0.215 | 97.13 |
| Compound 12 | DSPC | 123 | 0.22 | 97.58 |

### Example 10: Experiment on In Vivo Delivery of Luciferase mRNA in Animals

The nucleic acid delivery efficiency of lipid nanoparticles was characterized by observing the fluorescence expression in mice. The Luc-eGFP mRNA-encapsulated lipid nanoparticles prepared in Example 9 were injected at a certain dose into mice via the tail vein. Female C57/BL6 mice weighing 18 to 22 g were randomly grouped, with two mice in each group. After acclimation, Luc-eGFP mRNA-encapsulated LNPs (in a sterile PBS solution) were injected via the tail vein at a dose of 0.5 mg/kg (0.5 mg here referring to the dose of mRNA). In order to observe the expression of luciferase, the luciferase substrate was dissolved into a sterile PBS solution and prepared into a solution at a concentration of 30 mg/ml.

Six hours after injection of LNPs, 150 ul of luciferase substrate solution was injected intraperitoneally into each mouse. The mice were allowed to stand for 5 min, and then anesthetized for 3 min in a carbon dioxide box. After anesthesia, the mice were put in a small animal in vivo imager for imaging.

TABLE 2
Detection of Chemiluminescence Intensity from
Luc-eGFP mRNA Delivered by Lipid Nanoparticles

| Ionizable Lipid | Helper Lipid | Luminous Intensity 6 h (p/s/cm²/sr) |
|---|---|---|
| Compound 1 | DSPC | 3.4E+8 |
| Compound 1 | DOPE | 1.6E+9 |
| Compound 11 | DSPC | 7.2E+7 |
| Compound 12 | DSPC | 3.1E+8 |
| MC3 | DSPC | 1.35E+8 |

### Example 11: Animal Experiment—Subretinal Injection in Mice

The nucleic acid delivery efficiency of lipid nanoparticles was characterized by observing the fluorescence expression in mice. The lipid nanoparticles were prepared at a molar ratio of ionizable lipid (Compound 17, Compound 15, Compound 18, Compound 2 or MC3):DSPC:cholesterol:DSPE-PEG2000=50%:10%:39.5%:0.5%. Female C57/BL6 mice weighing 18 to 22 g were selected. After acclimation, the Luc-eGFP mRNA-encapsulated lipid nanoparticles were injected subretinally into mice (both eyes) at a dose of 0.9 μg (±10%) mRNA (1 μl, in a sterile PBS solution) per eye. In order to observe the expression of luciferase, the luciferase substrate was dissolved into a sterile PBS solution and prepared into a solution at a concentration of 30 mg/ml.

Imaging in mice: 18 hours after drug administration, 150 μl of luciferase substrate solution was injected intraperitoneally into each mouse. The mice were allowed to stand for 5 min, and then anesthetized for 3 min in a carbon dioxide box. After anesthesia, the mice were put in a small animal in vivo imager for imaging.

TABLE 3
Particle Sizes and Nucleic Acid Encapsulation Efficiencies of
Lipid Nanoparticles Containing Different Ionizable Lipids

| | Particle | | Nucleic Acid |
|---|---|---|---|
| Ionizable | Size | | Encapsulation |
| Lipid | nm | PDI | Efficiency (%) |
| Compound 17 | 90.31 | 0.36 | 99.4 |
| Compound 15 | 149.7 | 0.22 | 98.2 |
| Compound 18 | 121.7 | 0.14 | 96.0 |
| Compound 2 | 147.2 | 0.19 | 99.3 |
| MC3 | 149.0 | 0.24 | 96.6 |

TABLE 4
Detection of Chemiluminescence Intensity from Luc-eGFP
mRNA Delivered Subretinally by Lipid Nanoparticles

| Ionizable Lipid | Luminous Intensity 18 h (p/s/cm²/sr) |
|---|---|
| Compound 17 | 3.4E+8 |
| Compound 15 | 2.6E+7 |
| Compound 18 | 1E+7 |
| Compound 2 | 2E+8 |
| MC3 | 5E+6 |

What is claimed is:

1. A compound represented by formula (1) or a pharmaceutically acceptable salt thereof: 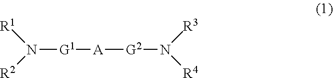
wherein
A is —O—CO—NH— or —NH—CO—O—;
G¹ and G² are independently C₁₋₄ alkylene;
R¹ and R² are independently -G³-O—C(═O)—R⁵, -G³-C(═O)—O—R⁵, -G³-NH—C(═O)—O—R⁵, -G³-NH—O—C(═O)—R⁵, -G³-NH—C(═O)—R⁵ or -G³-O—C(═O)—NH—R⁵;
G³ is independently C₂₋₁₀ alkylene;
R⁵ is C₂₋₃₀ alkyl or C₂₋₃₀ alkenyl, and said alkyl or alkenyl is optionally substituted with one or more of —O—C₁₋₃₀ alkyl, —C(═O)—O—C₁₋₃₀ alkyl, —O—C(═O)—C₁₋₃₀ alkyl, —O—C₂₋₃₀ alkenyl, —C(═O)—O—C₂₋₃₀ alkenyl or —O—C(═O)—C₂₋₃₀ alkenyl; and
R³ and R⁴ are independently C₁₋₈ alkyl.

2. The compound or the pharmaceutically acceptable salt thereof according to claim 1, wherein
G¹ and G² are independently —CH₂—, —CH₂CH₂—, —CH₂CH₂CH₂— or —CH₂CH₂CH₂CH₂.

3. The compound or the pharmaceutically acceptable salt thereof according to claim 1, wherein
R¹ and R² are independently -G³-O—C(═O)—R⁵, -G³-C(═O)—O—R⁵, -G³-NH—C(═O)—O—R⁵ or -G³-O—C(═O)—NH—R⁵;
G³ is independently C₂₋₈ alkylene;
R⁵ is C₂₋₂₀ alkyl or C₂₋₂₀ alkenyl, and said alkyl or alkenyl is optionally substituted with one or more of —O—C₁₋₂₀ alkyl, —C(═O)—O—C₁₋₂₀ alkyl, —O—C(═O)—C₁₋₂₀ alkyl, —O—C₂₋₂₀ alkenyl, —C(═O)—O—C₂₋₂₀ alkenyl or —O—C(═O)—C₂₋₂₀ alkenyl.

4. The compound or the pharmaceutically acceptable salt thereof according to claim 1, wherein
R¹ and R² are independently

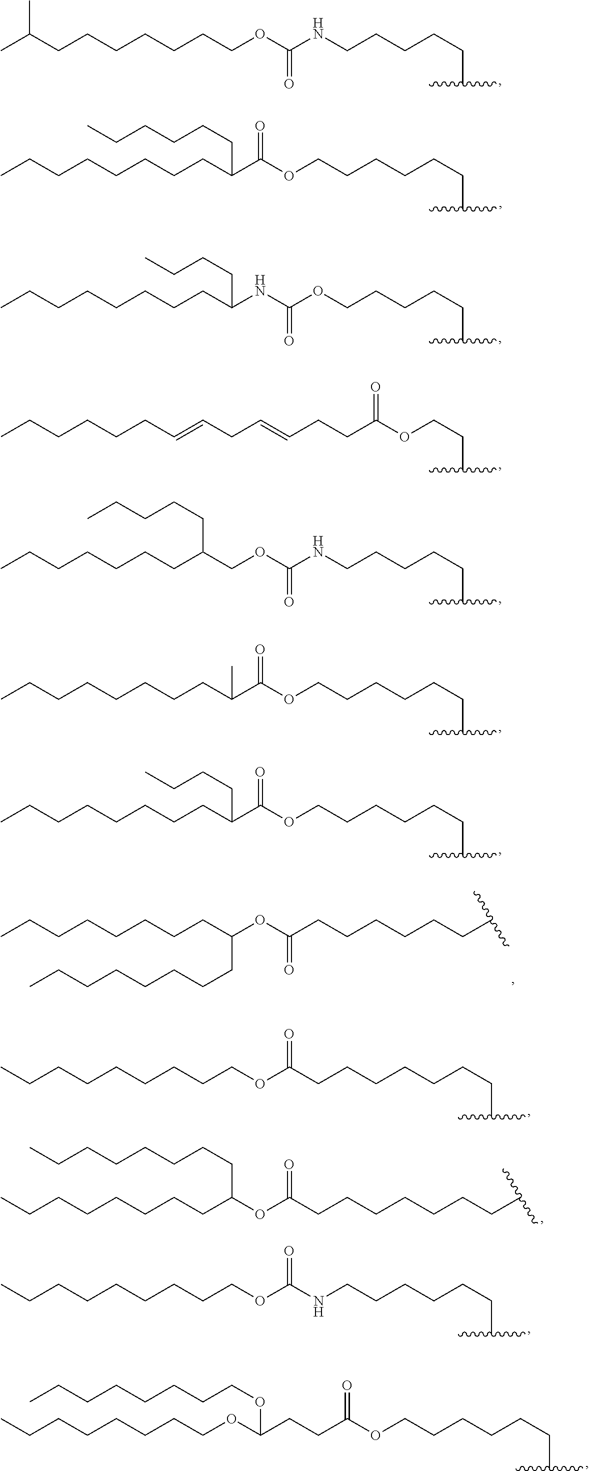

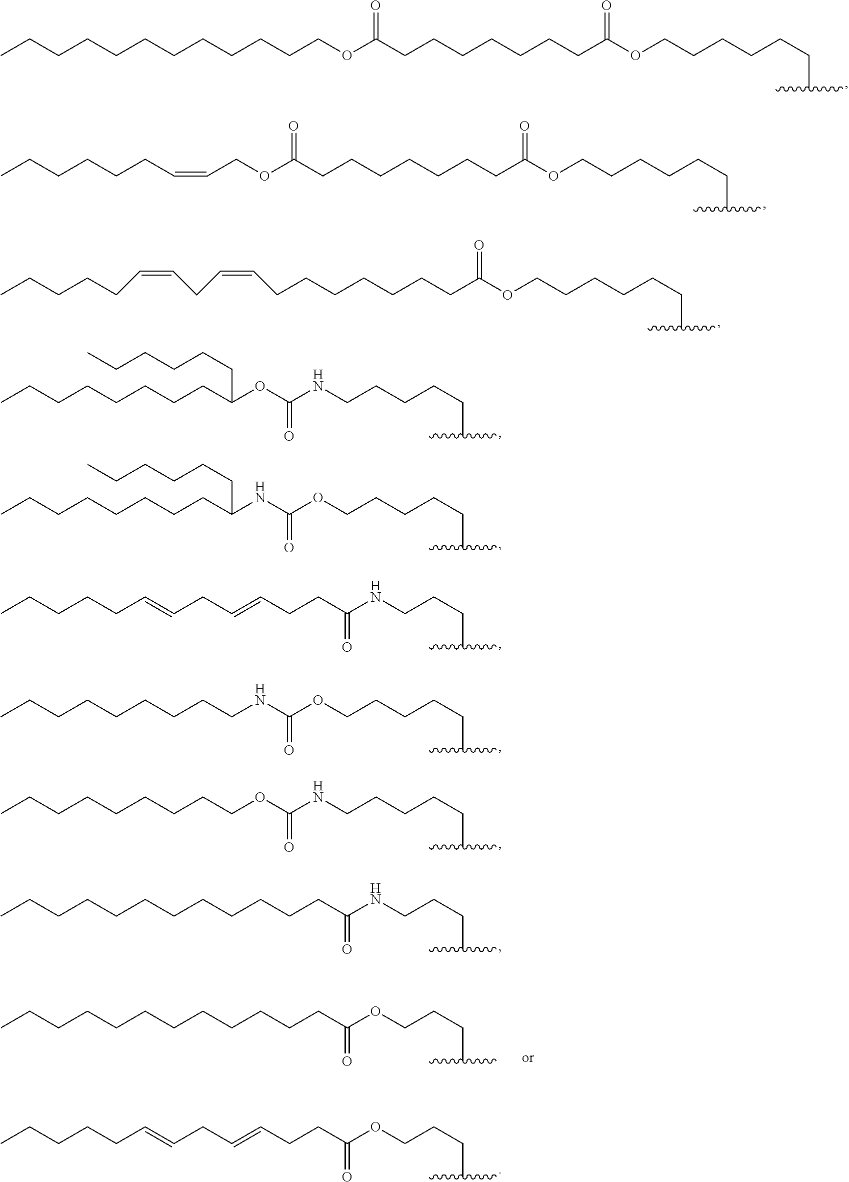

5. The compound or the pharmaceutically acceptable salt thereof according to claim 1, wherein
R³ and R⁴ are independently C₁₋₆ alkyl.

6. A compound or a pharmaceutically acceptable salt thereof, wherein the compound is selected from the following compounds:

| | Structure of Compound |
|---|---|
| 1 | 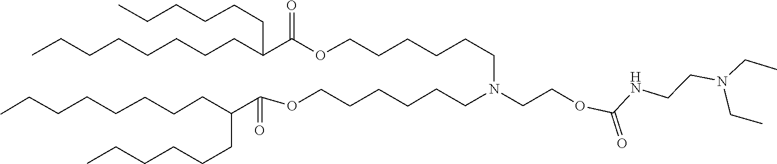 [SMILES: CCCCCCCCC(CCCCCC)C(=O)OCCCCCCN(CCCCCCOC(=O)C(CCCCCC)CCCCCCCC)CCOC(=O)NCCN(CC)CC] |
| 2 | 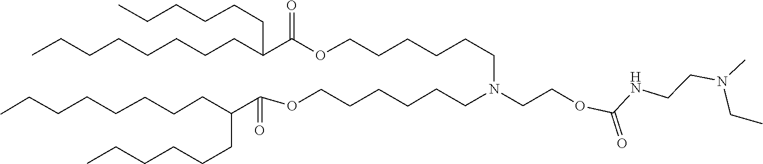 [SMILES: CCCCCCCCC(CCCCCC)C(=O)OCCCCCCN(CCCCCCOC(=O)C(CCCCCC)CCCCCCCC)CCOC(=O)NCCN(C)CC] |
| 3 | 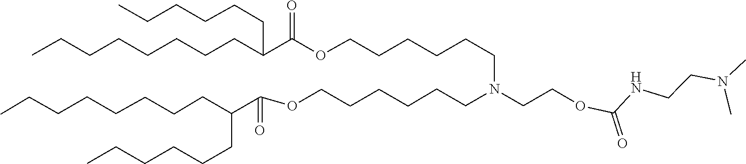 [SMILES: CCCCCCCCC(CCCCCC)C(=O)OCCCCCCN(CCCCCCOC(=O)C(CCCCCC)CCCCCCCC)CCOC(=O)NCCN(C)C] |
| 4 | 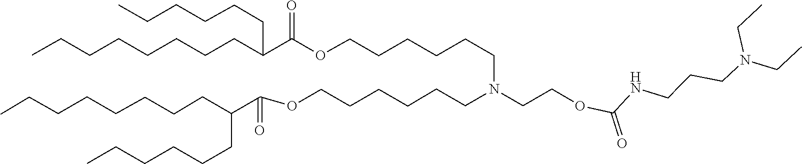 [SMILES: CCCCCCCCC(CCCCCC)C(=O)OCCCCCCN(CCCCCCOC(=O)C(CCCCCC)CCCCCCCC)CCOC(=O)NCCCN(CC)CC] |
| 5 | 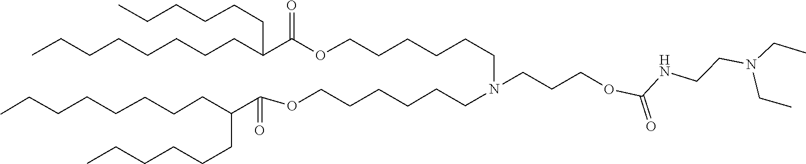 [SMILES: CCCCCCCCC(CCCCCC)C(=O)OCCCCCCN(CCCCCCOC(=O)C(CCCCCC)CCCCCCCC)CCCOC(=O)NCCN(CC)CC] |
| 6 | 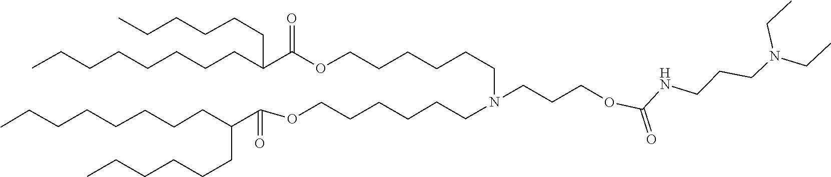 [SMILES: CCCCCCCCC(CCCCCC)C(=O)OCCCCCCN(CCCCCCOC(=O)C(CCCCCC)CCCCCCCC)CCCOC(=O)NCCCN(CC)CC] |
| 7 | 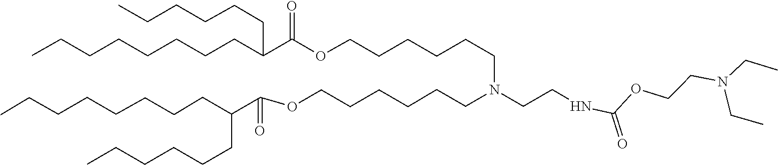 [SMILES: CCCCCCCCC(CCCCCC)C(=O)OCCCCCCN(CCCCCCOC(=O)C(CCCCCC)CCCCCCCC)CCNC(=O)OCCN(CC)CC] |
| 8 | 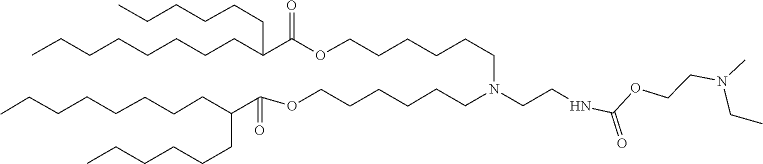 [SMILES: CCCCCCCCC(CCCCCC)C(=O)OCCCCCCN(CCCCCCOC(=O)C(CCCCCC)CCCCCCCC)CCNC(=O)OCCN(C)CC] |
| 9 | 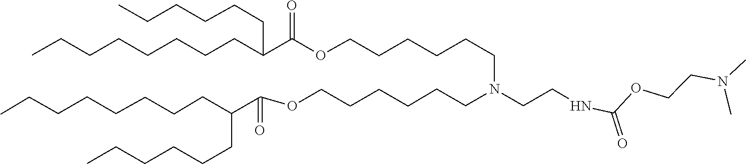 [SMILES: CCCCCCCCC(CCCCCC)C(=O)OCCCCCCN(CCCCCCOC(=O)C(CCCCCC)CCCCCCCC)CCNC(=O)OCCN(C)C] |
| 10 | 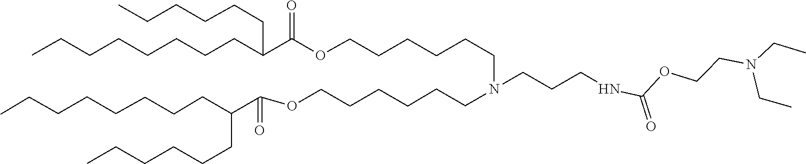 [SMILES: CCCCCCCCC(CCCCCC)C(=O)OCCCCCCN(CCCCCCOC(=O)C(CCCCCC)CCCCCCCC)CCCNC(=O)OCCN(CC)CC] |
| 11 | 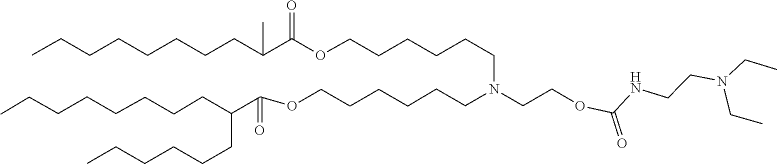 [SMILES: CCCCCCCCC(C)C(=O)OCCCCCCN(CCCCCCOC(=O)C(CCCCCC)CCCCCCCC)CCOC(=O)NCCN(CC)CC] |
| 12 | 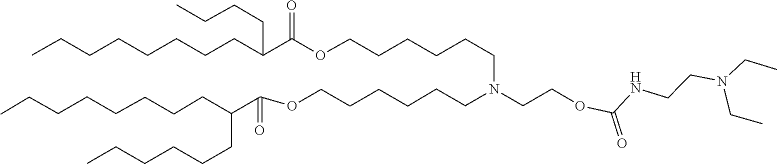 [SMILES: CCCCCCCCC(CCCC)C(=O)OCCCCCCN(CCCCCCOC(=O)C(CCCCCC)CCCCCCCC)CCOC(=O)NCCN(CC)CC] |
| 13 | 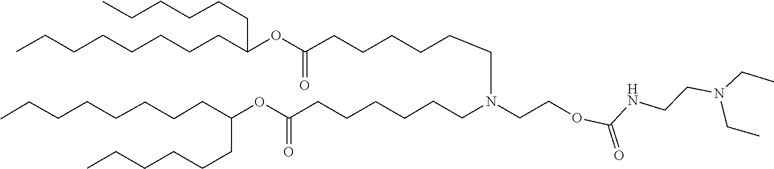 [SMILES: CCCCCCCCC(CCCCCC)OC(=O)CCCCCCN(CCCCCCC(=O)OC(CCCCCC)CCCCCCCC)CCOC(=O)NCCN(CC)CC] |
| 14 | 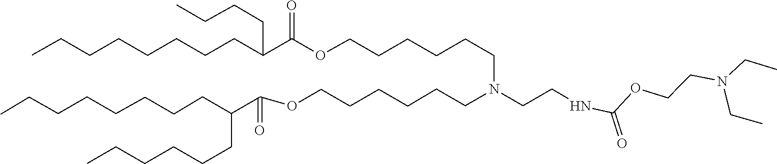 [SMILES: CCCCCCCCC(CCCC)C(=O)OCCCCCCN(CCCCCCOC(=O)C(CCCCCC)CCCCCCCC)CCNC(=O)OCCN(CC)CC] |
| 15 | 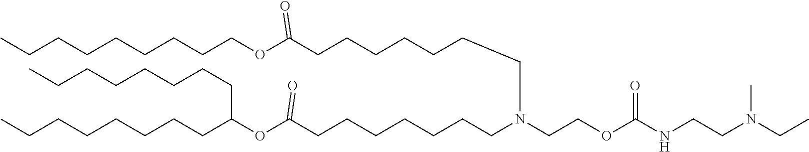 [SMILES: CCCCCCCCCOC(=O)CCCCCCCN(CCCCCCCC(=O)OC(CCCCCCCC)CCCCCCCC)CCOC(=O)NCCN(C)CC] |
| 16 | 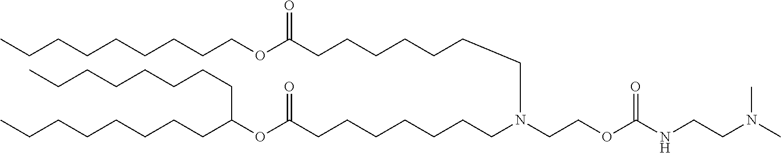 [SMILES: CCCCCCCCCOC(=O)CCCCCCCN(CCCCCCCC(=O)OC(CCCCCCCC)CCCCCCCC)CCOC(=O)NCCN(C)C] |
| 17 | 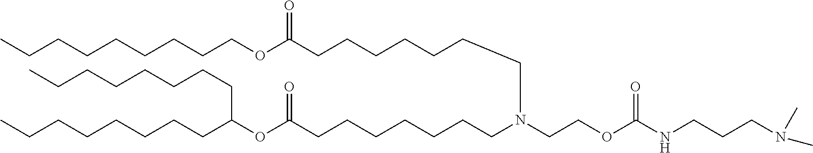 [SMILES: CCCCCCCCCOC(=O)CCCCCCCN(CCCCCCCC(=O)OC(CCCCCCCC)CCCCCCCC)CCOC(=O)NCCCN(C)C] |
| 18 | 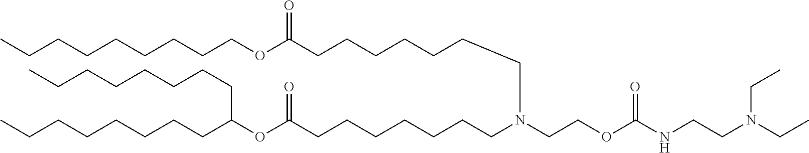 [SMILES: CCCCCCCCCOC(=O)CCCCCCCN(CCCCCCCC(=O)OC(CCCCCCCC)CCCCCCCC)CCOC(=O)NCCN(CC)CC] |
| 19 | 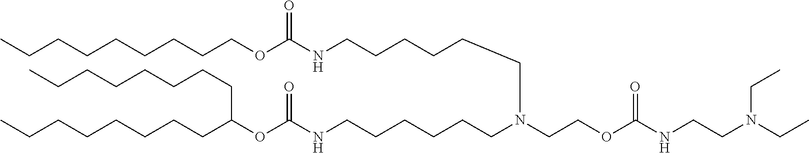 [SMILES: CCCCCCCCCOC(=O)NCCCCCCN(CCCCCCNC(=O)OC(CCCCCCCC)CCCCCCCC)CCOC(=O)NCCN(CC)CC] |
| 20 | 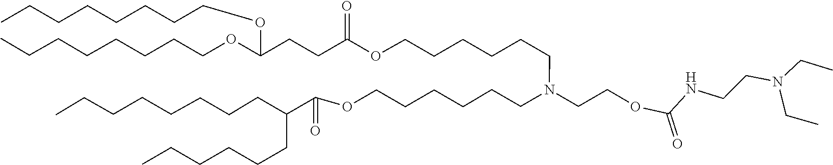 [SMILES: CCCCCCCCOC(CCC(=O)OCCCCCCN(CCCCCCOC(=O)C(CCCCCC)CCCCCCCC)CCOC(=O)NCCN(CC)CC)OCCCCCCCC] |
| 21 | 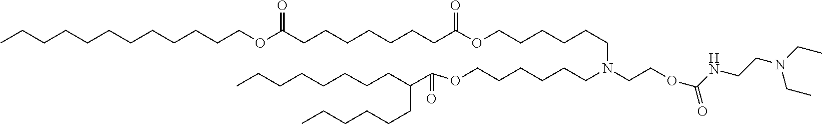 [SMILES: CCCCCCCCCCCCOC(=O)CCCCCCCC(=O)OCCCCCCN(CCCCCCOC(=O)C(CCCCCC)CCCCCCCC)CCOC(=O)NCCN(CC)CC] |
| 22 | 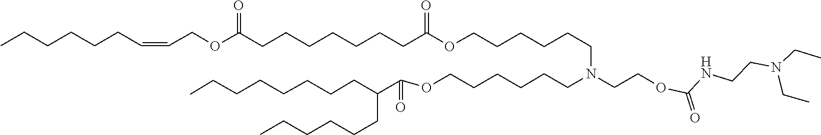 [SMILES: CCCCCC/C=C\COC(=O)CCCCCCCC(=O)OCCCCCCN(CCCCCCOC(=O)C(CCCCCC)CCCCCCCC)CCOC(=O)NCCN(CC)CC] |
| 23 | 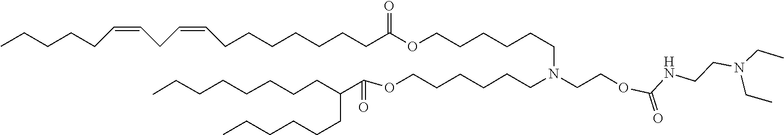 [SMILES: CCCCC/C=C\C/C=C\CCCCCCCC(=O)OCCCCCCN(CCCCCCOC(=O)C(CCCCCC)CCCCCCCC)CCOC(=O)NCCN(CC)CC] |
| 24 | 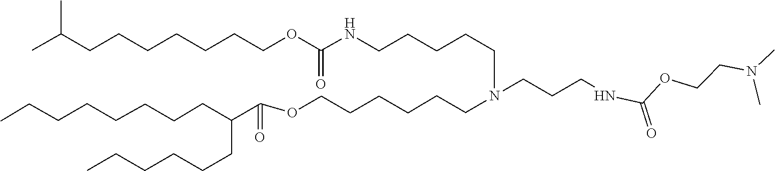 [SMILES: CCCCCCCCC(CCCCCC)C(=O)OCCCCCCN(CCCCCNC(=O)OCCCCCCCC(C)C)CCCNC(=O)OCCN(C)C] |
| 25 | 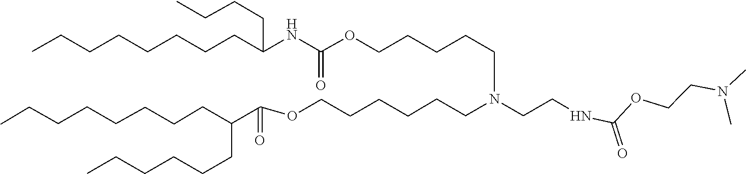 [SMILES: CCCCCCCCC(CCCC)NC(=O)OCCCCCN(CCCCCCOC(=O)C(CCCCCC)CCCCCCCC)CCNC(=O)OCCN(C)C] |
| 26 | 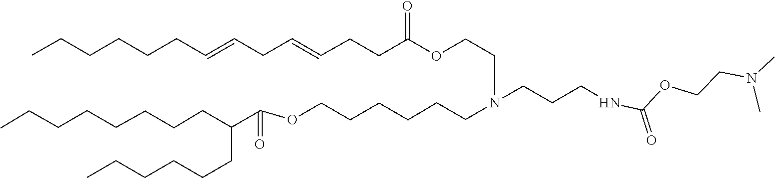 [SMILES: CCCCCC/C=C/C/C=C/CCC(=O)OCCN(CCCCCCOC(=O)C(CCCCCC)CCCCCCCC)CCCNC(=O)OCCN(C)C] |
| 27 | 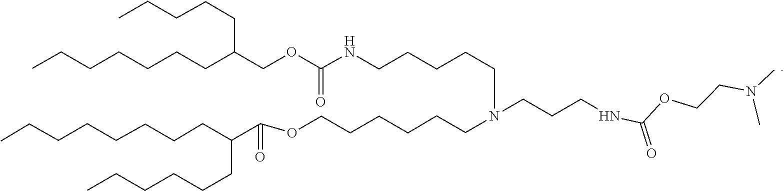 [SMILES: CCCCCCCCC(CCCCCC)C(=O)OCCCCCCN(CCCCCNC(=O)OCC(CCCCC)CCCCCCC)CCCNC(=O)OCCN(C)C] |

7. A lipid carrier, comprising the compound or the pharmaceutically acceptable salt thereof according to claim 1 as an ionizable lipid.

8. The lipid carrier according to claim 7, further comprising a helper lipid, a structural lipid, and a polymer-conjugated lipid.

9. A nucleic acid lipid nanoparticle composition, comprising the lipid carrier according to claim 7, and a nucleic acid.

10. The nucleic acid lipid nanoparticle composition according to claim 9, wherein the nucleic acid is at least one selected from DNA, RNA, a complex containing DNA or RNA, a modified DNA, a modified RNA, and a modified complex containing DNA or RNA.

11. A pharmaceutical preparation, comprising the nucleic acid lipid nanoparticle composition according to claim 9, and a pharmaceutically acceptable carrier.

12. A method for in vivo delivery of a nucleic acid, the method comprising administering to a subject in need thereof the nucleic acid lipid nanoparticle composition according to claim 9.

13. The compound or the pharmaceutically acceptable salt thereof according to claim 1, wherein
G³ is independently 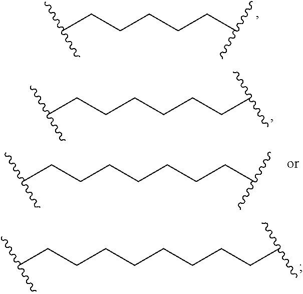 [SMILES: *CCCCC* ; *CCCCCC* ; *CCCCCCC* ; *CCCCCCCC*]
R⁵ is C₂₋₂₀ alkyl or C₂₋₂₀ alkenyl, and said alkyl or alkenyl is optionally substituted with one or more of —O—C₁₋₂₀ alkyl, —C(═O)—O—C₁₋₂₀ alkyl or —C(═O)—O—C₂₋₂₀ alkenyl; and
R³ and R⁴ are independently C₁₋₄ alkyl.

14. The compound or the pharmaceutically acceptable salt thereof according to claim 1, wherein
R⁵ is 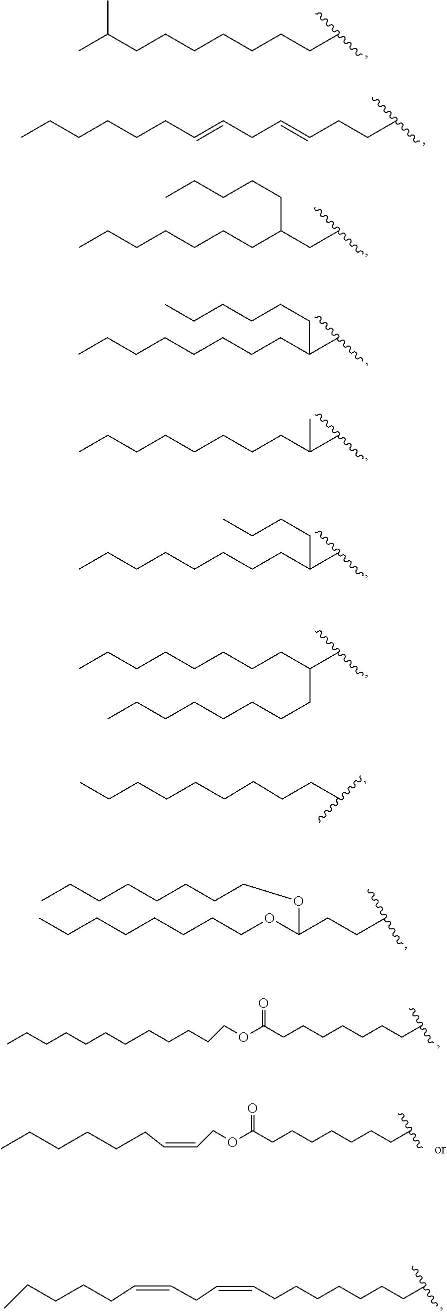
and
R³ and R⁴ are independently methyl or ethyl.

15. A lipid carrier, comprising the compound or the pharmaceutically acceptable salt thereof according to claim 6 as an ionizable lipid.

16. The lipid carrier according to claim 15, further comprising a helper lipid, a structural lipid, and a polymer-conjugated lipid.

17. A nucleic acid lipid nanoparticle composition, comprising the lipid carrier according to claim 15, and a nucleic acid.

18. The nucleic acid lipid nanoparticle composition according to claim 17, wherein the nucleic acid is at least one selected from DNA, RNA, a complex containing DNA or RNA, a modified DNA, a modified RNA, and a modified complex containing DNA or RNA.

19. A pharmaceutical preparation, comprising the nucleic acid lipid nanoparticle composition according to claim 17, and a pharmaceutically acceptable carrier.

20. A method for in vivo delivery of a nucleic acid, the method comprising administering to a subject in need thereof the nucleic acid lipid nanoparticle composition according to claim 17.
